(19) United States
(12) Patent Application Publication
(10) Pub. No.: US 2026/0241059 A1
(43) Pub. Date: Aug. 20, 2026

(54) AIR CLEANER

(71) Applicant: Omachron Intellectual Property Inc., Hampton (CA)

(72) Inventor: Wayne Ernest Conrad, Hampton (CA)

(21) Appl. No.: 19/640,948

(22) Filed: Apr. 7, 2026

Related U.S. Application Data
(63) Continuation of application No. 17/924,549, filed on Apr. 28, 2021, now abandoned.
(60) Provisional application No. 63/065,683, filed on Aug. 14, 2020.
(60) Provisional application No. 63/029,138, filed on May 22, 2020.

(51) Int. Cl. A61L 9/22

(52) U.S. Cl. CPC A61L 9/22

(57) ABSTRACT

A method for removing contaminants from air in a physical space. When a person is present, a person’s head is positioned between a lower band level, in which the person’s head is positioned above the lower band level, and an upper band level, in which the person’s head is positioned at least partially below the upper band level during normal use of the physical space. The method comprises providing negatively charged ions in the physical space; operating a positively charged ion collector to produce an electrical field having an electrical field strength, wherein the negatively charged ions in the band are drawn towards the positively charged ion collector due to a potential difference between the negatively charged ions and the positively charged ion collector; and adjusting the electrical field strength whereby negatively charged ions travel to the positively charged ion collector regardless of the airflow in the physical space.

### CROSS-REFERENCE TO RELATED APPLICATIONS

This patent application is a continuation of United States Patent Application No. 17/924,549, filed on November 10, 2022 as a national phase entry of International Patent Application No. PCT/CA2021/050581, filed on April 28, 2021, which itself claims the benefit of priority from United States Provisional Patent Application No. 63/065,683, filed on August 14, 2020 and United States Provisional Patent Application No. 63/029,138, filed on May 22, 2020, the contents of each of which are herein incorporated by reference.

### FIELD

This disclosure relates generally to an air cleaning system which may be used to filter biological contaminants from the air, such as a virus.

The following is not an admission that anything discussed below is part of the prior art or part of the common general knowledge of a person skilled in the art.

Various types of air cleaning systems. Typically, an air cleaning system uses air flow to move air through a filter media.

The following introduction is provided to introduce the reader to the more detailed discussion to follow. The introduction is not intended to limit or define any claimed or as yet unclaimed invention. One or more inventions may reside in any combination or sub-combination of the elements or process steps disclosed in any part of this document including its claims and figures.

In one aspect of the air cleaning system disclosed herein, which may be used by itself or with one or more other aspects disclosed herein, an air cleaning system uses a negative ion generator and a positively charged collector in an enclosed space, or in a partially enclosed space. The negative ion generator releases negative ions, which attach to contaminants and/or airborne particles (e.g., droplets in air exhaled by a person) in the enclosed space. Due to the release of the negative ions, an electrical potential difference is created between the negatively charged contaminants and the positively charged collection plate such that the negatively charged contaminants move towards and optionally attach to the collector.

An advantage of this design is that forced air flow through a filter is not required to remove contaminants from a volume of a room in which they may be inhaled by a person in the room. Instead, the negative ions attach to contaminants in the room, and, once negatively charged, the negatively charged contaminants move towards the collector. Another advantage of this design is that the potential difference between the negative ions and the collector may be varied based on several factors, such as, the size and shape of the room, and the desired risk level of contamination. For example, if the room has a high risk of contaminants, such as in a hospital or if the room may have a high load of people, the potential difference may be increased to accelerate the rate of removal of contaminants using the air cleaning system. Further, a sensor, such as an audio sensor or a sensor monitoring the number of people entering or exiting a room, may be used to monitor the number of people in a room and the rate of production of negative ions and/or the charge of the collector may be varied to respond to an increase in the number of persons in a room.

Another advantage of this design is that the air cleaning system is adaptable for each enclosed space. For example, in a conference room, several negative ion generators may be placed on a conference table with the collector located on the ceiling. In another example, in a school classroom, a negative ion generator may be placed on each student’s desk with the collector located on the ceiling.

Another advantage of this design is that occupants of the enclosed space can be within the electric field created by the electrical potential difference between the negative ions and the positive collector. Allowing an occupant to reside within the electric field may allow for improved safety of the occupant due to the rapid removal of contaminants from near the occupant.

Optionally, the negative ion generator includes an air moving member and/or a heat source to produced or enhance the air flow and/or convection in the enclosed space such that the negative ions emitted from the negative ion generator are distributed, e.g., throughout the enclosed space. An advantage of this embodiment is that the rate of removal of contaminants from the air may be increased due to the wider distribution of negative ions.

It will be appreciated that the negative ions may be provided only in a volume in which biological contaminants are anticipated. For example, in the case of a conference room, people will either be standing or sitting. Therefore, the negative ions may only be provided in the volume of the room in which people will exhale. Assuming that the people in a room with be of average height, then the negative ions may only be provided in a volume extending from, e.g., 3 feet above the floor to 6 or 7 feet above the floor.

Optionally, a person may carry a negative ion generator which may be in addition to or in lieu of one or more negative ion generators provided in the space, such as on a fixed or mobile platform.

In another aspect, a person may carry a negative ion generator as well as a positively charged collector. An advantage of this design is that the person may be in any location, which may be an enclosed space, a partially enclosed space or an open space, and may therefore have an apparatus which allows their face to be located within an electric field that is created by the electrical potential difference between the negative ions and the positive collector as the move about. The negative ion generator and positive collector plate may be one or more wearables, e.g., a wrist band, a lanyard, a hat, a visor, etc.

In accordance with these broad aspects, there is provided a physical space in which at least one person may be present wherein, when the person is in the physical space, the head of the person is positioned in a band extending from a lower band level, wherein the head of the person is positioned above the lower band level during normal use of the physical space, and an upper band level, wherein the head of the person is positioned at least partially below the upper band level during normal use of the physical space, the physical space comprising an apparatus for removing contaminants from air in a physical space, the apparatus comprising:

a) a first negative ion generator providing negative ions in the band; and,

b) a positively charged ion collector positioned outside the band,

In any embodiment, the apparatus may further comprise an air moving member operable to produce an air flow in the physical space, the air flow may be resolvable into a first flow vector directed at the one positively charged ion collector and a second flow vector directed away from the one positively charged ion collector, wherein a force imparted to the air by the second flow vector may be less than the field strength of the attractive force.

In any embodiment, the first negative ion generator may be positioned between the air moving member and the positively charged ion collector whereby the air flow may be at least substantially directed towards the positively charged ion collector such that the first flow vector comprises at least 70% of the momentum imparted to the air by the air moving member.

In any embodiment, the apparatus may further comprise an air moving member operable to produce an air flow in the physical space, the air flow having a velocity and a direction of flow wherein the velocity and direction of the air flow may be selected such that the air flow does not prevent the negatively charged ions travelling to the positively charged ion collector.

In any embodiment, the air flow may be directed towards the positively charged ion collector.

In any embodiment, the positively charged ion collector may comprise a positively charged member positioned interior of a dielectric member.

In any embodiment, an air gap may be positioned between the positively charged member and the dielectric member.

In any embodiment, the positively charged ion collector may have a smooth outer surface.

In any embodiment, the positively charged ion collector may comprise a self-disinfecting member which treats an outer surface of the positively charged ion collector whereby viruses on the outer surface are denatured.

In any embodiment, the first negative ion generator may be a wearable.

In any embodiment, the first negative ion generator may be provided on a mobile autonomous robot.

In any embodiment, the first negative ion generator may be fixedly mounted to a location in the physical space and the apparatus may further comprise a second negative ion generator that may be mobile in the physical space.

In any embodiment, the first negative ion generator and the positively charged ion collector may be mounted on a mobile device.

In any embodiment, at least one of the first negative ion generator and the positively charged ion collector may be adjustable to maintain a generally constant field strength as the location of the person in the physical space varies.

In any embodiment, the physical space may comprise a conference room and the first negative ion generator may be provided at a level of a table in the conference room and the positively charged ion collector may be provided on the ceiling.

In any embodiment, the positively charged ion collector may be part of a light fixture.

In any embodiment, the physical space may comprise a work space and the first negative ion generator may be provided at a level of a work surface in a work station and the positively charged ion collector may be provided above the upper band level in the work station.

In any embodiment, the work space may comprise a work station in an office, a bank teller station or a check out station in a store.

In any embodiment, the negative ion generator may be positioned in front of a person while working at the work station and the positively charged ion collector may be positioned overlying the person while working at the work station.

In any embodiment, the apparatus may further comprise an additional positively charged ion collector that may be provided below the lower band level in the work station.

In another aspect, a method is provided for operating an electric filed using any embodiment of the apparatus that is discussed herein.

In accordance with another aspect, the collector of the air cleaning system may be self-cleaning and/or self-disinfecting and/or may be made of a material that has an activity that will denature a biological contaminant. For example, the collector may include a disinfecting module that disinfects at least a portion of the collector during and/or after use.

It will be appreciated that the electric filed may be generated in an open space, such as an open air stadium or the like. For example, in an open air stadium, there may be little or minimal air movement due to wind and accordingly, the stadium may be provided with positive collector plates and the negative ion generators may be one of more of a wearable carried by a person, a negative ion generator provided at a fixed location and a mobile negative ion generator (e.g., a negative ion generator provided on a self-propelled autonomous apparatus, e.g., a robot).

It will be appreciated by a person skilled in the art that an apparatus or method disclosed herein may embody any one or more of the features contained herein and that the features may be used in any particular combination or sub-combination.

These and other aspects and features of various embodiments will be described in greater detail below.

### BRIEF DESCRIPTION OF THE DRAWINGS

For a better understanding of the described embodiments and to show more clearly how they may be carried into effect, reference will now be made, by way of example, to the accompanying drawings in which:

FIG. 1 is a perspective view of a first embodiment of an air cleaning system having a negative ion generator and a collector;

FIG. 2 is a cross-sectional view of the negative ion generator of FIG. 1 along the line 2-2;

FIG. 3 is a side view of the air cleaning system of FIG. 1, illustrating an electric field;

FIG. 4 is a side view of the air cleaning system of FIG. 1;

FIG. 5 is a perspective view of another embodiment of an air cleaning system;

FIG. 6 is a front view of another embodiment of an air cleaning system;

FIG. 7 is a front view of an embodiment of a rotatable collector with a disinfecting module;

FIG. 8A is a front view of another embodiment of a rotatable collector with a disinfecting module;

FIG. 8B is a top view of the collector of FIG. 8A;

FIG. 9 is a perspective view of another embodiment of an air cleaning system;

FIG. 10 is a side view of another embodiment of an air cleaning system located in a restaurant;

FIG. 11 is a side view of another embodiment of an air cleaning system located in an enclosed space with high ceilings;

FIG. 12 is a side view of an embodiment of a plurality of ionizing sources;

FIGS. 13A-13C are side views of embodiments of air moving members at varying angles in an air cleaning system;

FIGS. 14A-14C are cross-sectional views of embodiments of positive collectors with varying insulators;

FIGS. 15A-15B are a side view and a front view, respectively, of another embodiment of an air cleaning system;

FIG. 16 is a side view of another embodiment of an air cleaning system with a plurality of air moving members;

FIG. 17 is a side view of another embodiment of an air cleaning system with a wearable negative ion generator;

FIGS. 18A-18C are perspective views of various embodiments of an air cleaning system;

FIG. 19 is a perspective view of another embodiment of an air cleaning system;

FIG. 20 is a perspective view of another embodiment of an air cleaning system;

FIG. 21 is a perspective view of another embodiment of an air cleaning system;

FIG. 22 is a top view of the air cleaning system of FIG. 21;

FIG. 23 is a perspective view of another embodiment of an air cleaning system; and,

FIG. 24 is a perspective view of an embodiment of a wearable air cleaning system.

The drawings included herewith are for illustrating various examples of articles, methods, and apparatuses of the teaching of the present specification and are not intended to limit the scope of what is taught in any way.

### DESCRIPTION OF EXAMPLE EMBODIMENTS

Various apparatuses, methods and compositions are described below to provide an example of an embodiment of each claimed invention. No embodiment described below limits any claimed invention and any claimed invention may cover apparatuses and methods that differ from those described below. The claimed inventions are not limited to apparatuses, methods and compositions having all of the features of any one apparatus, method or composition described below or to features common to multiple or all of the apparatuses, methods or compositions described below. It is possible that an apparatus, method or composition described below is not an embodiment of any claimed invention. Any invention disclosed in an apparatus, method or composition described below that is not claimed in this document may be the subject matter of another protective instrument, for example, a continuing patent application, and the applicant(s), inventor(s) and/or owner(s) do not intend to abandon, disclaim, or dedicate to the public any such invention by its disclosure in this document.

The terms "an embodiment," "embodiment," "embodiments," "the embodiment," "the embodiments," "one or more embodiments," "some embodiments," and "one embodiment" mean "one or more (but not all) embodiments of the present invention(s)," unless expressly specified otherwise.

The terms "including," "comprising" and variations thereof mean "including but not limited to," unless expressly specified otherwise. A listing of items does not imply that any or all of the items are mutually exclusive, unless expressly specified otherwise. The terms "a," "an" and "the" mean "one or more," unless expressly specified otherwise.

As used herein and in the claims, two or more parts are said to be “coupled”, “connected”, “attached”, or “fastened” where the parts are joined or operate together either directly or indirectly (i.e., through one or more intermediate parts), so long as a link occurs. As used herein and in the claims, two or more parts are said to be “directly coupled”, “directly connected”, “directly attached”, or “directly fastened” where the parts are connected in physical contact with each other. None of the terms “coupled”, “connected”, “attached”, and “fastened” distinguish the manner in which two or more parts are joined together.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures, and components have not been described in detail so as not to obscure the example embodiments described herein. Also, the description is not to be considered as limiting the scope of the example embodiments described herein.

As used herein, the wording “and/or” is intended to represent an inclusive - or. That is, “X and/or Y” is intended to mean X or Y or both, for example. As a further example, “X, Y, and/or Z” is intended to mean X or Y or Z or any combination thereof.

Airborne particles and/or contaminants may result in detrimental health effects and/or infection of humans. Biological contaminants, such as those released by coughing, sneezing, and breathing, may be filtered or removed from a physical space to reduce the risk of contamination. When people are located outdoors, the risk of contamination/infection from airborne contaminants is likely lower than the risk of contamination/infection in an enclosed space due to the contaminants being entrapped in the enclosed space. Enclosed spaces often have limited air flow, thereby limiting the ability of natural air flow to reduce the risk of contamination from airborne contaminants by removing the contaminants from the enclosed space. Limited air flow may result in the entrapment and/or build-up of contaminants and debris. Accordingly, air cleaning systems may be used to filter, remove, or reduce contaminants and debris that are released in enclosed spaces. Conventional air cleaning systems make use of fans to create air flows that travel in particular circulation patterns, allowing contaminants to pass through filters or outside of the enclosed space. Air flows in enclosed spaces may be interrupted or interfered with by occupants of the room such that they may take a long time to clean all of the air in the room. The use of filters requires that the contaminated air flows pass through the filter itself to clean the air. However, filter air cleaning systems are often inefficient, since a volume of air equal to several times the volume of an enclosed space must pass through the filtration member to treat all or substantially all of the air in an enclosed space. Accordingly, filter air cleaning systems often treat the same air that has already passed through the filtration member, resulting in an extended operating time to treat most or all of the air in the enclosed space. Depending on the situation, a more rapid and efficient air cleaning system may improve the safety and/or health of occupants in the enclosed space. For example, if biological contaminants are released into an enclosed space by, e.g., an infected person, rapid removal of the particles from the air in the enclosed space may protect the other occupants in the room by reducing the likelihood of the other occupants inhaling the biological contaminants expelled by an infected person. Another example is in the case of a fire. Rapid air cleaning of smoky air may protect or even save the lives of the occupants of the room.

In order to reduce the risk of exposure of undesirable contaminants to an occupant in a physical space, an air cleaning system using a high electrical potential difference may be used. Such a high potential difference air cleaning system includes at least one negative ion generator (and optionally a plurality of negative ion generators) and at least one positively charged collector (and optionally a plurality of positively charged collectors).

The negative ions created by the negative ion generator attach to contaminants and/or airborne particles in the room, including droplets containing biological contaminants such as viruses, providing the contaminants with a negative charge. The electrical potential difference between the negatively charged contaminate and the positively charged collector produces an attractive force having a field strength, which causes the negatively charged contaminants to move towards and optionally attach to the collector.

The collector may be disinfected or may include a material that kills or denatures the contaminants. By strategically positioning the negative ion generator(s) and the collector(s) in the enclosed space, the risk of exposure to the contaminants in the enclosed space may be limited or eliminated.

Additionally, the use of negative ions provides a safe method of removing contaminants from an enclosed space. Studies have shown that negative ions in the air have no detrimental effects on humans, and may provide positive benefits to humans.

In order to treat the air in the enclosed space, the air cleaning system requires a sufficient potential difference between the negatively charged particles and the positively charged collector to reduce the level of contaminants to an acceptable degree of risk in an acceptable period of time. The higher the potential between the contaminants and the collector, the faster the contaminants will move to the collector. In other words, the potential difference (without any induced air flow or convective flow) may by itself create a sufficient driving force such that the contaminants, which have been negatively charged by the negative ions released from the negative ion generator, will be attracted towards the positively charged collector at a sufficient rate so as to remove a sufficient quantity of contaminants from the volume of air in the enclosed space that a person may inhale so that the amount of biological contaminants remaining is reduced or substantially reduced so as to reduce the likelihood that a person that inhales air in the enclosed space may be infected and, optionally, the amount of biological contaminants remaining may be reduced such that there is an insufficient concentration of contaminants remaining to cause infection to an occupant of the enclosed space. For example, if the contaminants are biological, such as a virus, removal of a sufficient quantity of the biological contaminate may be in the range of 99% or higher in order to inhibit or prevent an infected person infecting another occupant of the enclosed space. It will be appreciated that, if a room is vacant, a lower potential difference may be used, which will remove contaminants at a slower rate due to the lower potential difference creating a lower driving force which thereby causes negatively charged particles to move at a slower rate to the positive collector(s). If the room is occupied, the potential difference is optionally higher to create a larger driving force.

Various factors may be adjusted to provide a desired treatment efficiency. These include, but are not limited to, the amount of negative ions produced per unit time, the level of positive charge of the collector, the number of negative ion generators, the number and size of positive collectors, the distance between the positive collector and the negative ion generator and/or the velocity of air flow within the air cleaning system.

During use, the negative ion generator generates and releases negative ions into the air. In order to accelerate the treatment of an enclosed space, a large number of negative ions may be produced per unit time. The rate at which the negative ion generator emits negative ions may vary according to use. If a higher contaminant loading is expected, or if there is a higher loading of people in an enclosed space, then the rate of production of negative ions may be increased. For example, in some embodiments, the negative ion generator may emit negative ions at a rate in the range of 10x10¹³ to 8x10¹⁵ ions per second.

The amount of negative ions produced by a negative ion generator will vary depending upon the charge of the negative ion emitter. The higher the negative charge that is applied, the higher the amount of negative ions which may be produced per unit time. For example, a negative charge in the range of -1,000V to -25,000V, -2,000V to – 25,000V, -4,000V to – 15,000V, -5,000V to -10,000V, or -6,500 to -10,000V, or any included range may be used.

It will be appreciated that ozone may be produced at higher negative voltages. Therefore, in order to reduce, or prevent or essentially prevent, the formation of significant amounts of ozone, a larger number of negative ion generators, which operate at a lower negative voltage, may be operated to produce a desired amount of ions. For example, two or more, 5 or more, or 10 or more negative ion generators may be used in an enclosed space.

Alternately, or in addition, in order to accelerate the treatment of an enclosed space, one or more collectors having a higher positive charge may be used and/or a larger number of positively charged collectors may be used and/or the positively charged collector(s) may have a larger surface area and/or the position of the positively charged collector(s) may be adjusted to position the positively charged collector(s) closer to the source of contaminants.

The charge of the collector may be varied to alter the potential difference produced by the air cleaning system. For example, in some embodiments, the collector may be charged in the range of 2,000V to 1,000,000V, 10,000V to 500,000V, 40,000V to 200000V, or 50,000V to 100,000V, or any included range. It will be appreciated that these ranges may vary greatly depending on the use of the air cleaning system.

Another factor that may affect the treatment efficiency is the distance between the ion generator and the collector. The greater the distance between the ion generator and the collector, the larger the potential difference that is required to have the same treatment efficiency, all other factors remaining the same. For example, if the negative ion generator is on the floor and the collector is mounted to a ceiling, the potential may need to be greater than if the negative ion generator were placed on a table and the collector is mounted to a wall at a lower height than the ceiling.

Another factor that may affect the sufficient potential value is the average distance between occupants in the enclosed space. For example, if two occupants are standing within three feet of one another, the potential difference may be lower than if the two people were standing within one foot of each other. The closer people stand or sit to each other in the enclosed space, the faster biological contaminants need to be removed in order to lower the risk of infection to a reasonable degree.

Another factor that may affect the potential difference is the degree of risk of contamination and/or infection in the enclosed space. For example, if the enclosed space is a hospital room with known infected patients, the potential difference is likely much higher than if the enclosed space is a dentist’s office or a restaurant or a bar. Accordingly, the rate of removal may be increased by increasing the potential difference, to reduce the risk of exposure/infection to a reasonable degree.

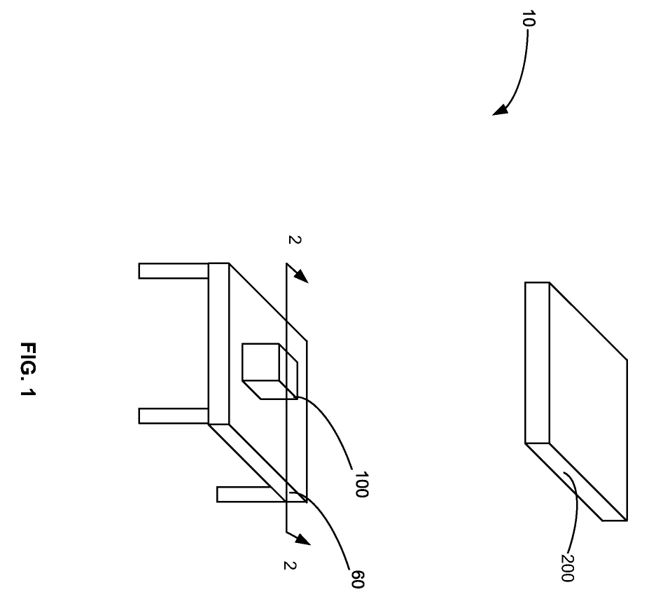

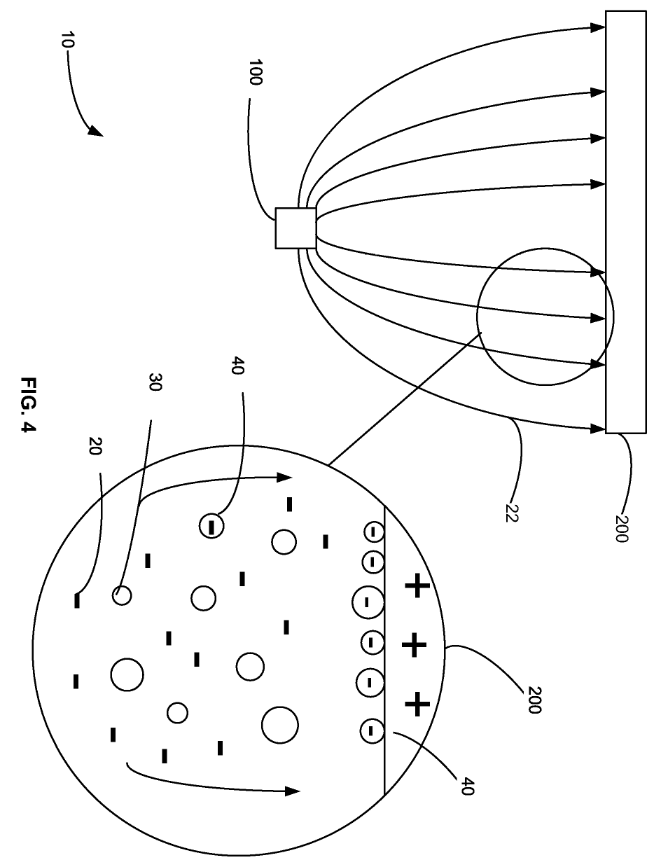

In accordance with this aspect, which may be used by itself or in combination with one or more other aspects, there is provided an apparatus for removing contaminants from air in a physical space, also referred to as an air cleaning system 10, as exemplified in FIG. 1. The air cleaning system 10 comprises a negative ion generator 100 and a positively charged collector 200. As described above, the negative ion generator 100 generates and releases ions 20 into the air. The air in the enclosed space may have contaminants and/or airborne particles, generally referred to as contaminants 30. The negative ions 20 attach to and charge the contaminants 30, thereby creating negatively charged contaminants 40. The contaminants 40 move towards and attach to the collector 200, as exemplified in FIG. 4.

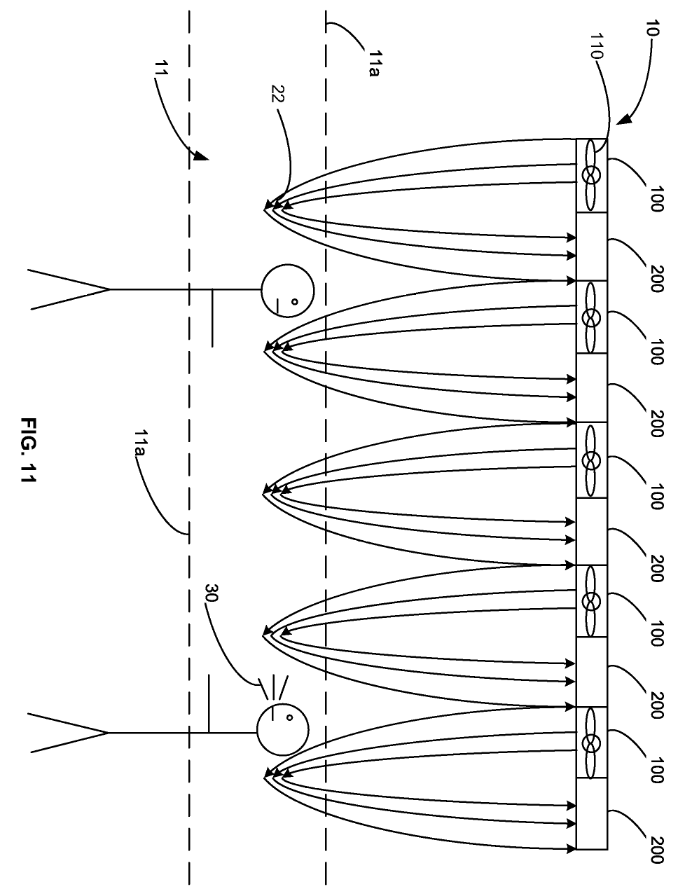

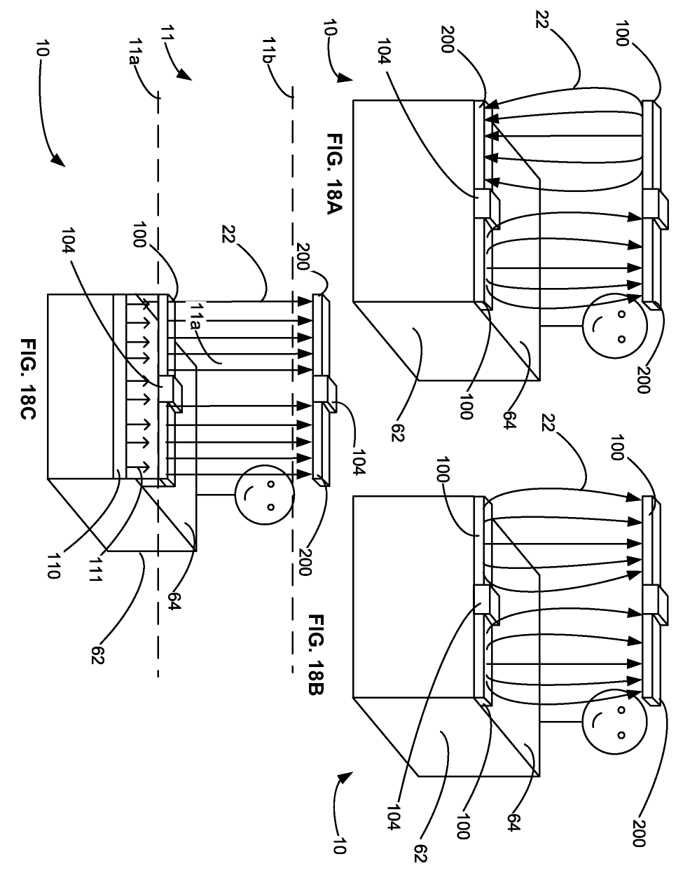

It will be appreciated that the enclosed space including the air cleaning system 10 may be partially enclosed. For example, in some embodiments, the air cleaning system 10 may be used with umbrellas at restaurant patios, awnings, gazebos, garages, pavilions, open air stadiums etc. For simplicity, the area in which the air cleaning system 10 will be referred to as an enclosed space, however, it should be understood that the air cleaning system 10 may be used in any indoor or outdoor area that has a risk of airborne contaminate build-up and/or entrapment. In other words, the air cleaning system 10 may be used in any physical space in which at least one person may be present. It will be appreciated that the direction and location of the release of negative ions 20 into the physical space may vary depending on the desired use. For example, when the person is located in the physical space, the head of the person may be positioned in a band 11, as exemplified in FIGS. 11 and 18C. The band 11 has a lower band level 11aand an upper band level 11b. As exemplified, the head of the person is positioned above the lower band level 11a during normal use of the physical space and the head of the person is positioned at least partially below the upper band level 11b during normal use of the physical space.

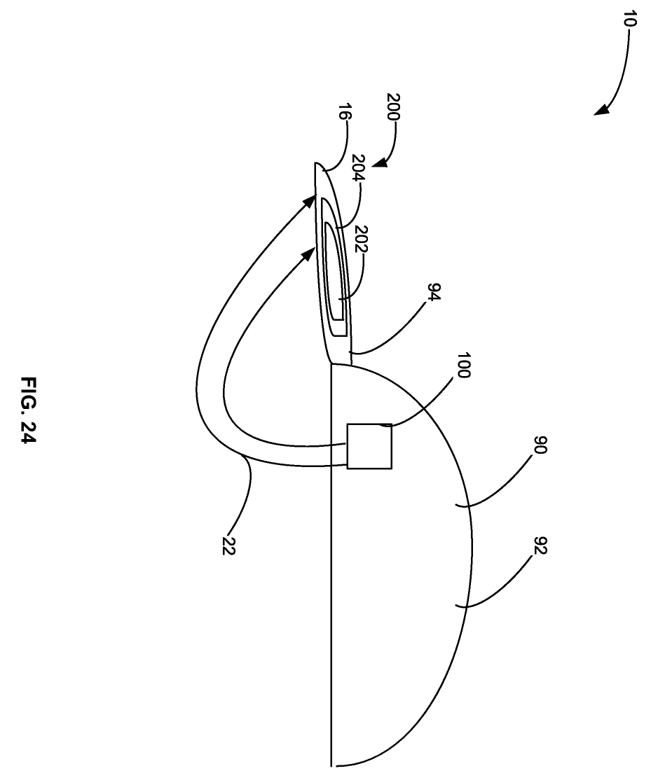

It will be appreciated that the negative ion generator 100 and the collector 200 may be positioned anywhere within the enclosed space. For example, in some embodiments, the negative ion generator 100 and positive collector 200 may be positioned outside the band, while in other embodiments they may be positioned within the band 11. In some embodiments, one of the positive collector 200 or negative ion generator 100 may be positioned within the band 11 while the other is positioned outside the band 11. As exemplified in FIG. 11, the positive collector 200 is positioned outside of the band 11. As exemplified, the negative ion generator 100 is also positioned outside the band 11, but is capable of providing negative ions 20 into the band 11. In another example, the negative ion generator may be a wearable that in use is located below a person’s face (e.g., a necklace) and the collector may be in a hat or, as exemplified in FIG. 24, they may each be provided in a hat.

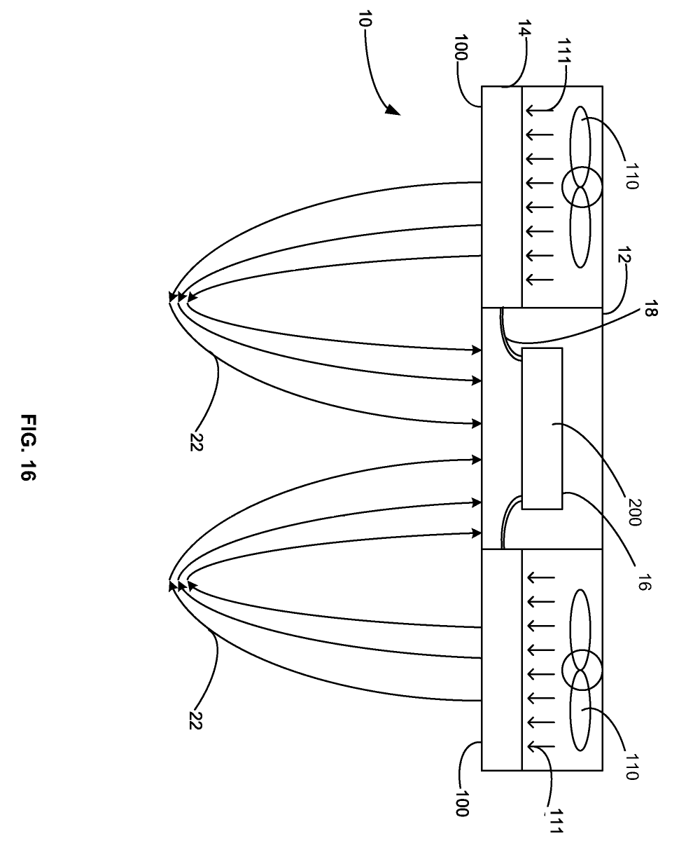

In some embodiments, for example, the negative ion generator 100 and the collector 200 may both be positioned within a system housing 12, as exemplified in FIG. 16. As exemplified, the system housing 12 encompasses both the negative ion generator 100 and the collector 200. As shown, the system housing 12 is rectangular in shape. It will be appreciated that the system housing 12 may be any shape and/or size. In some embodiments, the negative ion generator 100 may have a separate negative housing 14 and the collector 200 may have a separate positive housing 16, as exemplified in FIG. 1. In some embodiments, the negative housing 14 and the positive housing 16 may be coupled together or may be separate from one another. For example, as exemplified in FIG. 16, the system housing 12 includes the negative housing 14 and the positive housing 16.

It will be appreciated that the negative ion generator 100 may use any means to create negative ions. For example, the negative ion generator may use a negative electrode 102 shaped as a pin to generate negative ions 20. Similarly, it will be appreciated that the positive collector 200 may use any means to create a positive charge. For example, the positive collector 200 may have a positive electrode 202. It will be appreciated that the positive and negative electrodes may be formed of any material. For example, the electrodes may be formed of, including, but not limited to, nickel, stainless steel, and/or tungsten. In some embodiments, the positive collector 200 may use a transformer having primary coils and secondary coils to generate a positive voltage, such as a tesla coil. The details of the creation of the negative and positive charges will be discussed in more detail subsequently.

It will be appreciated that the power supply to the air cleaning system 10 may vary with the design of the system. For example, in some embodiments, a single power supply may be used to provide power to both the negative ion generator 100 and the collector 200, as exemplified in FIGS. 18A-18C. In some embodiments, the negative ion generator 100 may have a generator power supply and the collector 200 may have a collector power supply. In some embodiments, the generator power supply and the collector power supply may be connected together to create a neutral net charge (i.e. the charge of the negative ions that are produced balances the positive charge of the collector 200). As exemplified in FIG. 16, the collector 200 and the negative ion generators 100 are coupled together by an electrical connector 18.

It will be appreciated that the power supply to the negative ion generator 100 and/or to the collector 200 may be any means of providing power. In some embodiments, the power supply may be wired to the negative ion generator 100 and/or the collector 200. The power supply may be hard wired to one or both of the negative ion generator 100 and the collector 200. Such an embodiment may be used, e.g., in a conference room (see for example FIG. 1). In such a case, the source of power may be an electrical outlet in the room or the like. In such a case an uninterruptible power supply may be provided.

Alternately, negative ion generator 100 and/or the collector 200 may be electrically connectable to a power source. For example, the wired connection may use a USB-C connector in the housing of the system 10 and/or in the negative housing and the positive housing. In such a case, the one or both of the negative ion generator 100 and the collector 200 may have an onboard power supply (e.g., a battery or a capacitor) such that they may operate when disconnected from the power source.

In some embodiments, the power supplies may be wireless. For example, the power supply may use an inductive coupling to wireless power the negative ion generator 100 and/or the collector 200.

In some embodiments, the power supply may be an onboard power supply. Accordingly, when the power supply for the negative ion generator 100 and/or the collector 200 is a battery, the negative ion generator 100 and/or the collector 200 may be portable.

In accordance with an aspect, which may be used by itself r with any one or more other aspects set out herein, one or more of the housings may include one or more insulators to protect the air cleaning system 10 and the occupants in the enclosed space from the high voltage generated. For example, if the positive collector is highly charged, then one or more insulators may be provided.

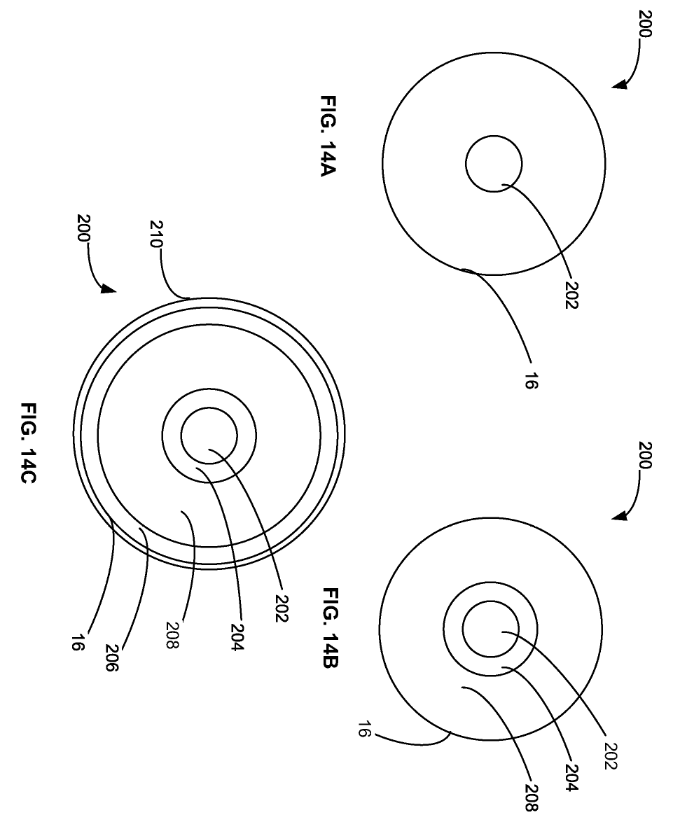

As exemplified in FIGS. 14A-14C, the positive electrode 202 of the collector 200 may be positioned interior of a dielectric member. For example, in some embodiments, a first insulator 204 may be applied over the primary coil of the collector 200, within the positive housing 16. The first insulator 204 may be any material capable of insulating the collector 200. For example, the first insulator may be any dielectric. By insulating the primary coils of the collector 200, the rest of the collector 200, the system 10, and/or the occupants of the enclosed space may be protected from the high voltage of the collector 200 should they come into contact with collector 200.

Optionally, the collector 200 may have a second insulator 206. It will be appreciated that the second insulator 206 may be applied over the first insulator 204 and/or any other component of the collector 200. The second insulator 206 may improve the insulation of the positive electrode 202 of the collector 200. The second insulator 206 may be the same material as the first insulator 204. Alternately, the second insulator 206 may be a different material than the first insulator 204. For example, the second insulator 206 may be made of a material that is resistant to chemical damage, such as cleaning chemicals. Forming the second insulator 206 of a material resistant to chemical damage may increase the lifetime of the collector 200. For example, the second insulator 206 may protect the first insulator 204 from chemical damage when the collector 200 is cleaned. Accordingly, the integrity of the insulators may be improved.

Optionally, at least one of the first or second insulators may be formed of a material that is resistant to degradation by ozone and/or oxides of nitrogen and/or sulfur. Optionally, the insulators may be resistant to degradation in both dry and moist conditions.

It will be appreciated that the first and second insulators may be in contact with one another or may be spaced apart from each other. For example, as exemplified in FIGS. 14A-14C, a gap insulator 208, which may be an air gap, may be provided. As exemplified, the air gap 208 may be between the first insulator 204 and the second insulator 206. It will be appreciated that, in some embodiments, the gap insulator 208 may be the only insulator used within the collector 200, as exemplified in FIG. 14A. Alternately a gap insulator 208 may be used if a single insulator 204, 206 is used (see for example FIG. 14B). It will be appreciated that the gap insulator 208 may include one or more gases. For example, the gap insulator 208 may include air or a noble gas such as argon or it may be a partial vacuum.

In some embodiments, the spacing between the first and second insulators may be at a distance greater than the discharge gap in air of the applied high voltage. In other words, the spacing may be great enough that even if a spark were generated by the primary coils, the spark may not travel the entire length of the spacing between insulators. Accordingly, the positive housing 16 of the collector 200 may be positioned at a distance such that it is past the dielectric breakdown of air, reducing the risk of shock, either by arcing or direct contact, to a user.

Alternately, or in addition, it will be appreciated that a secondary coil may be insulated. For example, the first insulator 204, the second insulator 206, and/or the gap insulator 208 may be used to insulate the secondary coil in the collector 200.

It will be appreciated that the negative ion generator 100 may have one or more insulators, as described above for the collector 200. It will also be appreciated that the negative ion generator insulators may be designed to release the generated negative ions 20. For example, the first insulator may have openings to allow negative ions 20 to pass from the negative generator into the gap insulator 208 (the air gap). The second insulator may also have openings to allow the negative ions 20 to be release from the negative housing. Accordingly, the second insulator may protect an occupant from directly contacting the negative ion generator 100. The air gap may protect an occupant by creating a large enough space between the occupant and the negative ion generator 100 that the occupant is protected from the spark gap region. Therefore, an occupant may be protected from arcing and direct contact with the negative ion generator 100, while still allowing the negative ion generator 100 to release negative ions 20 through the negative housing.

In some embodiments, the system housing 12, the negative housing 14, and/or the positive housing 16 may form the second insulator 206. For example, the first insulator may surround the negative electrode with the negative housing spaced apart from the first insulator. As described above, this spacing may allow negative ions 20 to be release from the negative ion generator 100, while also preventing an occupant from entering the discharge gap.

In accordance with an aspect, which may be used by itself r with any one or more other aspects set out herein, the casing enclosing an electrode may be sealed, e.g., it may be gas tight, dust tight and/or liquid tight.

For example, the housing 12 of the system 10, the negative housing 14, and/or the positive housing 16 may also be referred to as a shell. The shell may protect the power supply, the positive electrode, and/or the negative electrode from air, dust, water, or any other contaminate. For example, the shell may be gas tight, dust tight and/or liquid tight. Optionally, the air and/or oxygen may be purged from the shell before it is sealed gas tight, thereby reducing the risk of dielectric breakdown within the shell.

Optionally, the shell may be formed of the first insulator 204. Accordingly, the first insulator 204 may act as a seal against dust, air, and/or liquid. Alternately, the shell may be formed of the second insulator 206. For example, the electrode 202 of the collector 200 may be completed sealed by the second insulator 206. In some embodiments, there may be a plurality of shells. For example, the first insulator 204 may form a shell around the primary coils while the second insulator 206 may form a shell around the secondary coils of the collector 200. Each of the first and second shells may be gas tight, dust tight, and/or liquid tight.

It will be appreciated that the shell may surround any portion of the air cleaning system 10 to protect against air, dust, and/or liquid. For example, in some embodiments, the shell may surround the power supply to both the negative ion generator 100 and the collector 200. In some embodiments, the shell may surround the collector power supply and/or the generator power supply. In some embodiments, the shell may surround the positive electrode 202. In some embodiments, the shell may surround both the power supply 104 and the positive electrode 202.

In some embodiments, the shell may be removable from the air cleaning system 10. For example, the positive housing 16 may be removed from the collector 200 to facilitate cleaning of the collector 200. Accordingly, the shell may be openable (e.g., it may be formed of two halves which are rotatably connected together by a screw thread or a bayonet lock or they may be pivotally connected together).

It will be appreciated that the housings and/or shells may be formed of a smooth material to improve the cleaning of the air cleaning system 10. For example, reducing or eliminating seams in the housing and/or shells may reduce the build-up of contaminants on the surface of the housing and/or shells. Additionally, reduction or elimination of seams may reduce the likelihood of UV shadows. UV shadows are regions that are not reachable by UV light used for sanitizing a surface. Accordingly, the presence of UV shadows may reduce the ability of a surface to be properly sanitized.

As exemplified in FIG. 14C, a cover 210 may be positioned over the collector 200 to provide a surface to collect negatively charged contaminants 40. The shell and/or housing 16 may allow the cover 210 to be easily removed and/or cleaned without putting an occupant at risk within the spark gap. It will be appreciated that the cover 210 may be coupled to the collector 200 by any means. For example, the cover 210 may be attached by, including but not limited to, clips, magnets, semi-permanent adhesive, rubber bands, a hook and loop fastener, or any combination thereof.

In some embodiments, the shell may enclose the positive electrode 202 of the collector 200 and may be filled with a fluid to improve the lifespan and/or operation of the collector 200. For example, the shell may be filled with nitrogen, iron oxide, etc.

In accordance with this aspect one or both of the negative ion generator 100 and the positive collector 200 are configured, spaced and/or operated to reduce and, optionally, essential prevent the production of ozone and oxides of nitrogen, and/or oxides of sulfur, collectively referred to as byproducts of dielectric breakdown.

As described previously, the electrical potential difference between the negative ions 20 and the collector 200 may vary depending on a number of factors. The strength of the electric field is a function of the source charges and the distance between the two charges. Accordingly, the strength of the electric field may be increased by moving the negative ions 20 closer to the collector 200, increasing the magnitude of the voltage of the negative ion generator 100 and/or increasing the magnitude of the voltage of the collector 200.

The strength of the electric field between the negative ions 20 and the collector 200 is determined by the absolute value of the relative charges. For example, if the negative ions 20 are charged at -2000 kV and the collector 200 is charged at +10,000 kV, at the same distance, the strength of the electric field is stronger than if the charges were -1000 kV and +1000 kV respectively. It will be appreciated that the absolute value of the charges of the negative ions 20 and the collector 200 may be equal or may be different. For example, the negative ion 20 may have a larger absolute value charge than the collector 200, or the collector 200 may have a larger absolute value charge than the negative ions 20.

However, increasing the negative voltage of the negative ion generators 100 may result dielectric breakdown of the air in the enclosed space. Dielectric breakdown of air may result in the production of byproducts of dielectric breakdown. These byproducts may be detrimental to the health of occupants in the enclosed space. Accordingly, the voltage of the negative ion generators 100 may remain the same or may be lowered while the voltage of the positive collector 200 may be increased to increase the strength of the electric field.

While increasing the positive voltage of the collector 200 may reduce the likelihood of ozone creation by the negative ion generator 100, increasing the positive voltage of the collector 200 may also result in dielectric breakdown of air. Dielectric breakdown caused by the collector 200 may also create byproducts of dielectric breakdown. Accordingly, care can be taken to reduce the likelihood of dielectric breakdown caused by the positive voltage by lowering the positive voltage below a threshold value. However, in some embodiments, the positive voltage value may need to be above the positive threshold value in order to maintain a sufficiently strong electrical field without increasing the negative voltage of the negative ion generator 100 beyond its negative threshold value. Accordingly, alternately, or in addition, the risk of exposure of ozone and/or cations to occupants in the enclosed space may be reduced by fully encapsulating the positive electrode in the collector 200. As described above, the shell may completely encapsulate the collector 200, preventing cations and/or ozone from being released into the air in the enclosed space. However, depending on the shape of the shell and/or positive housing of the collector 200, ozone and/or cations may be produced outside of the shell and/or positive housing due to the high voltage values. That being said, the shape of the collector 200 may be designed to reduce the likelihood of dielectric breakdown outside of the housing and/or shell. Ozone and cations are more likely to be generated by wires and sharp edges. Accordingly, the shell and/or positive housing may be designed to have a generally round, smooth, shape to reduce the risk of ozone and/or cation production. The design of the collector 200 may be such that no ozone and/or cations are released into the air of the enclosed space. Specific structures of the collector 200 will be discussed subsequently.

For example, the shape of the positive electrode may be rounded (e.g., spherical, cylindrical or ellipsoid) in order to reduce the likelihood of dielectric breakdown. If the collector 200 is a plate, then the edges of the plate may be rounded.

Optionally, the shell may be formed of two or more pieces that are bonded together to secure the electrode inside the shell. For example, two half tubes may form the shell to surround the electrode. Forming the shell of two pieces may reduce manufacturing costs and/or time, as well as providing easier access to the electrode if the shell is openable. Additionally, bonding the two shell pieces together may reduce the number of edges in the shell, thereby reducing the likelihood of dielectric breakdown.

In some embodiments, the air cleaning system 10 may have a plurality of negative ion generators 100. As discussed subsequently, the negative ion generator may be a point source. The point sources may be spaced apart so as to reduce or essentially eliminate the production of byproducts of dielectric breakdown.

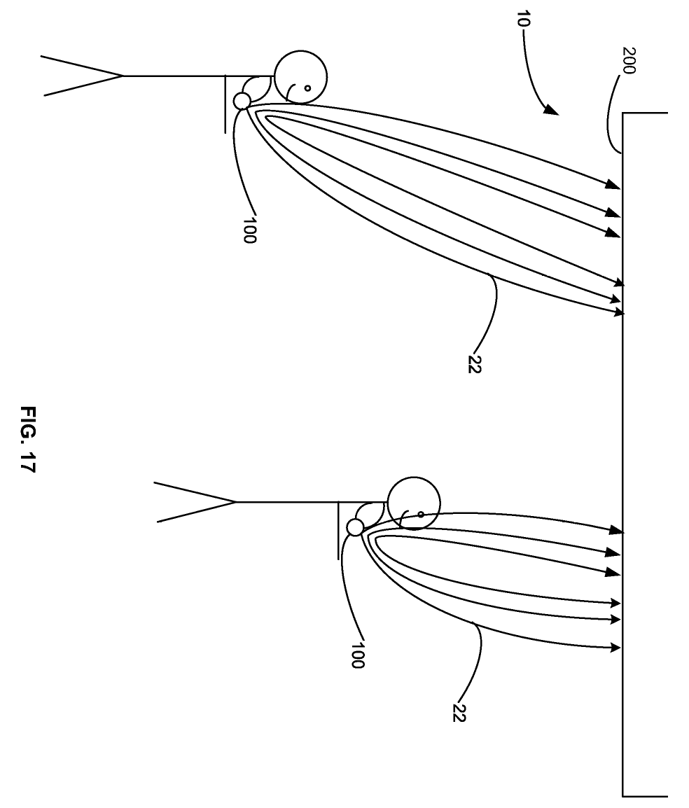

It will be appreciated that each of the negative ion generators 100 in the plurality of negative ion generators 100 may operate at the same voltage or may operate at different voltages. For example, since the electric field strength is a function of distance, negative ion generators 100 that are located further from the collector 200 may have a larger negative voltage than negative ion generators 100 located closer to the collector 200 in order to maintain a sufficient electrical potential difference in the air cleaning system 10, as exemplified in FIG. 17. As described above, the increase in negative voltage in the negative ion generator 100 may result in the dielectric breakdown of air and production of ozone and other byproducts. Accordingly, the negative ion generators 100 may be maintained at or below the negative voltage threshold value and the positive voltage value of the collector 200 may be adjusted to account for the varying differences in the plurality of negative ion generators 100 caused by the varying distances from the collector 200. As described above, the collector 200 may be designed such that no cation and/or ozone is released into the enclosed space, despite the increase in the positive voltage value used to compensate for the lower negative voltage of the negative ion generators 100. Accordingly, the strength of the electric field may be optimized while maintaining the safety of the occupants of the enclosed space. It will be appreciated that the field strength of the electric field may be adjustable by changing the voltage of the negative ion generator 100 and/or collector 200, by changing the position of the negative ion generator 100 and/or collector 200, or both.

In some embodiments, a plurality of positive collectors 200 may be used to improve the air cleaning system 10. Accordingly, the positive voltage of the collectors 200 may be reduced proportionately to the reduced distance to the negative ion generators. For example, when a negative ion generator 100 is positioned far enough away from a first collector 200, a second collector 200 may be positioned closer to the negative ion generator 100 to allow for the reduction in negative voltage without a reduction in the strength of the electric field.

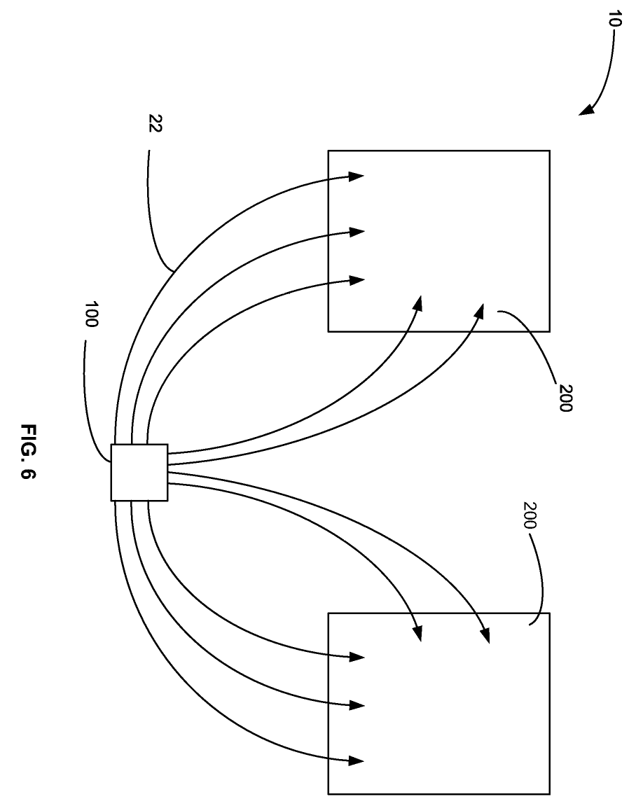

In accordance with this aspect, which may be used by itself or in combination with one or more other aspects, there is provided a negative ion generator 100 for use in the air cleaning system 10, as exemplified in FIGS. 1-13 and 15-24.

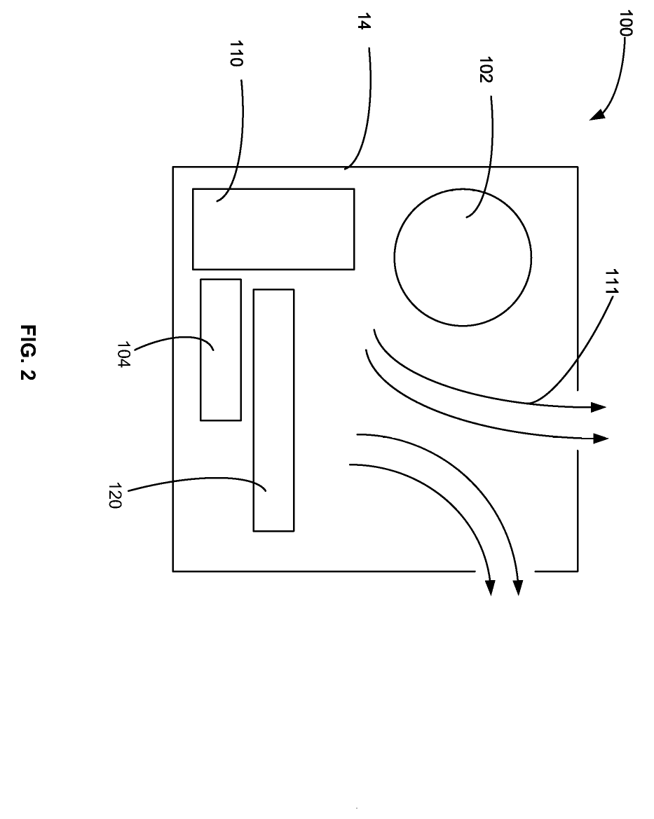

The negative ion generator 100 includes at least one ionizing source 102. The ionizing source 102 is connected to a power supply 104. It will be appreciated that the power supply 104 may be any means of providing power to the negative ion generator 100, including, but not limited to, AC power, DC power, or a battery. As exemplified in FIG. 2, the power supply 104 is an uninterruptible power supply such as a battery. The battery 104 may be charged through wired charging, such as by USB, or may be charged wirelessly, such as through induction charging, or by both wired or wireless charging. For example, the negative ion generator 100 may be designed to use induction charging on tables with built-in induction chargers, making it easy to add negative ion generators 100 to existing enclosed spaces.

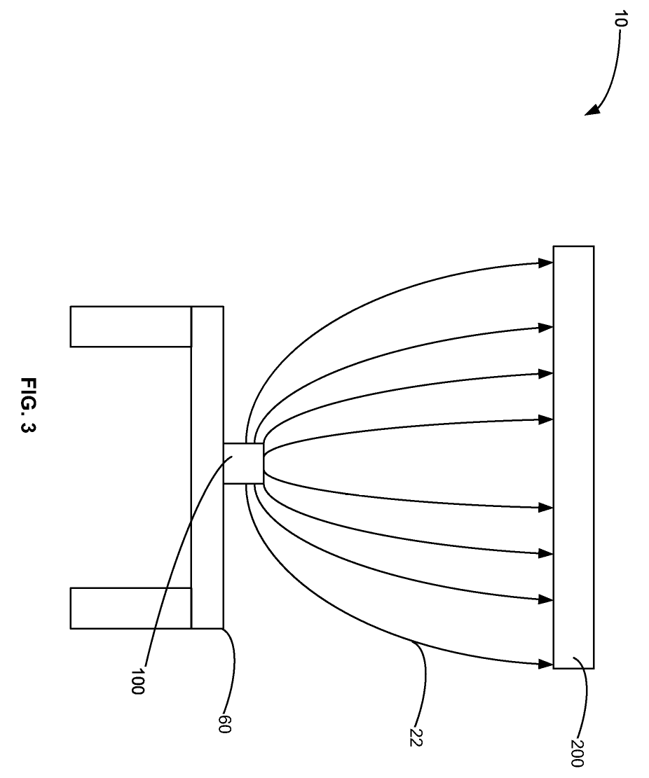

During use, the ionizing source 102 releases negative ions 20 into the air, negatively charging contaminants 30, thereby creating negatively charged contaminants 40. As illustrated in FIGS. 3 and 4, an electrostatic field 22 is created between the negatively charged contaminants 40 and the positively charged collector 200. As illustrated in FIG. 4, the electric potential difference between the positively charged collector 200 and the negatively charged contaminants 40 causes the negatively charged contaminants 40 to move and attach to the collector 200.

As described previously, the speed and efficacy of the air cleaning system 10 may be varied by changing the driving force driving the negatively charged particles 40 towards the collector 200. It will be appreciated that other means of improving the efficacy of the air cleaning system 10 may be used.

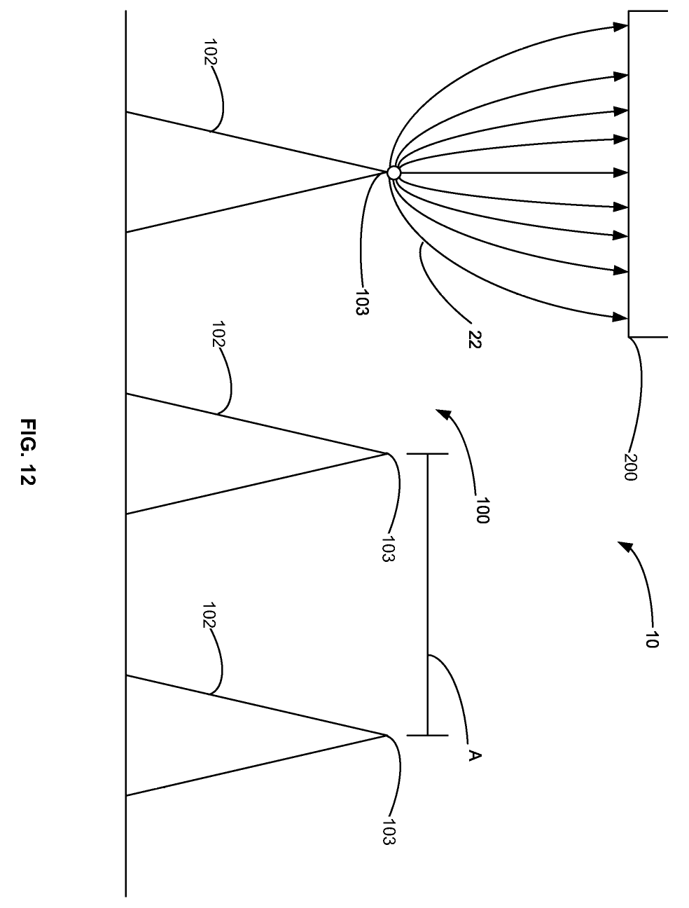

The negative ion generator 100 may produce a large quantity of negative ions very rapidly. As described previously, negative ions 20 may be produced at a rate in the range of 10x10¹³ to 8x10¹⁵ ions per second. To produce the negative ions 20, a negative voltage is applied to a negative electrode, referred to above as the ionizing source 102. When the negative voltage reaches a threshold negative value, negative ions 20 are released from the negative electrode. The greater the negative voltage, the more negative ions 20 that are released. To improve the efficiency at which negative ions 20 are released, the negative electrode may be shaped to have a very sharp tip 103, as exemplified in FIG. 12. The sharp tip 103 may increase the number of negative ions 20 that are produced.

As described previously, if the negative voltage applied to the ionizing source 102 is increased beyond a threshold value, dielectric breakdown of the air at the tip 103 of the ionizing source 102 may result in the production of ozone and/or oxides of nitrogen and/or sulfur. Accordingly, to reduce the likelihood of dielectric breakdown while maintaining the strength of the electric field, the negative voltage value may be lowered and the number of ionizing sources 102 may be increased. Increasing the number of ionizing sources 102 while reducing the applied negative voltage may result in an increase of produced negative ions 20 without a significant increase in ozone or other byproducts of dielectric breakdown.

In some embodiments, the ionizing source 102 may have a plurality of sharp tips to release more negative ions 20. However, clusters of sharp tips may result in a loss of efficacy in the production of negative ions due to the close proximity of multiple ionizing sources 102. As voltage increases, if the distance between the sharp tips 103 is too small, the electrostatic field 22 may encompass all of the sharp tips 103, in effect creating a single point source. The creation of a single point source may increase the production of ozone or other byproducts of dielectric breakdown. In other words, the negative voltage applied to a particular unit of air may increase due to the clustering of the negative ion sources 102. Increasing the negative voltage in a particular unit of air may result in a more rapid dielectric breakdown. Accordingly, the sharp tips may be spread out as point sources, or may be aligned in a linear fashion to improve the efficacy of negative ion production and/or reduce the creation of ozone. As exemplified in FIGS. 12, the tips 103 are a distance “A” from each other. For example, in some embodiments, the distance A between ionizing sources 102 may be 0.1”, optionally 0.25”, optionally 1”, optionally 2”, optionally 6”, or optionally 12”. It will be appreciated that the distance A between ionizing sources 102 may vary depending on the desired use of the air cleaning system.

In some embodiments, the negative ion generator 100 may include guides that extend adjacent to the sharp tips 103. The guides may provide electrical insulation between adjacent sharp tips 103 such that adjacent ionizing sources 102 operate as individual negative on generators and reducing the likelihood that adjacent ionizing sources 102 will operate as a single point source.

In some embodiments, a plurality of ionizing sources 102 may be patterned to improve the production of negative ions 20. For example, the ionizing sources 102 may be formed in concentric circles. The concentric circles may have sufficient spacing between them to improve the production of negative ions 20. Guides may separate the concentric circles, as described previously.

In accordance with this aspect, which may be used by itself or in combination with one or more other aspects, the negative ion generator 100 may use repulsion to help distribute negative ions 20 into the enclosed space. For example, the ionizing source 102 with the one or more sharp tips 103 may have a base that extends into the housing of the negative ion generator 100. Connecting the ionizing source 102 to the rest of the negative ion generator 100 may negatively charge the negative ion generator 100. Negatively charging the negative ion generator 100 may result in a repulsion force between the negative ion generator 100 and the generated negative ions 20. As the negative ions 20 are released, they may be repelled away from the negative ion generator 100 by the charge of the negative ion generator 100 itself.

Optionally, a conductive material may be used to negatively charge at least a portion of the negative ion generator 100. For example, one or more ionizing sources 102 may be coupled to a conductive material 106 within the negative ion generator 100. The conductive material 106 may be made of any material capable of holding a charge.

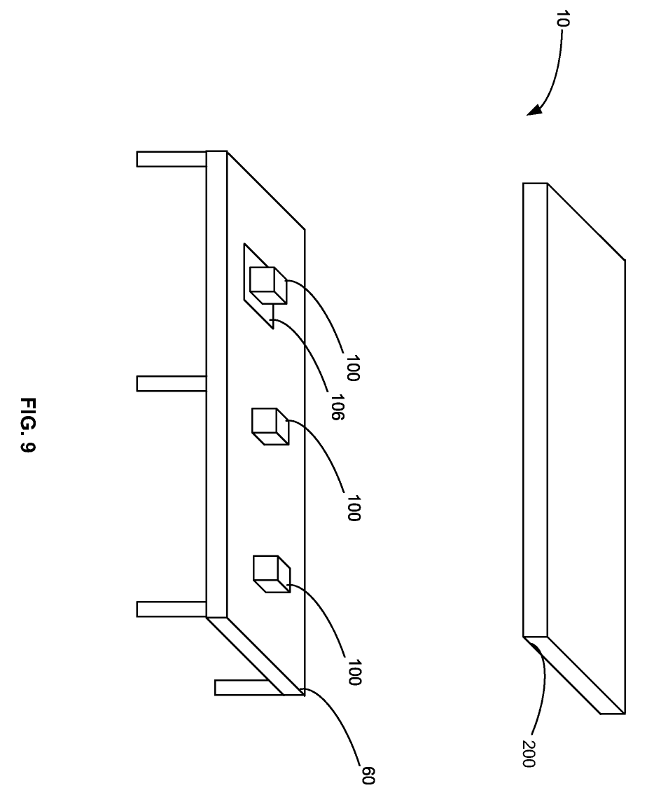

Alternately, or in addition, a negatively charged member may be provided adjacent or attached to the negative ion generator 100. for example, as exemplified in FIG. 9, conductive material 106 may extend beyond the housing of the negative ion generator 100. For example, the conductive material may form a table mat for use on a table. Alternately, or in addition, the conductive material 106 may extend beyond the negative housing to negatively charge a separate surface. For example, the negative ion generator 100 may be used to negatively charge a table, a person, a mat, a lamp, etc. The conductive material 106 may help direct released negative ions 20 upwards away from the table, to improve the negative charging of contaminants 30. To reduce the likelihood of ozone creation, the conductive material may be formed of a woven or nonwoven material.

In accordance with another aspect, that may be used by itself or in combination with one or more other aspects, the negative ion generator 100 may generate air flow and/or forced convection in the enclosed space. An advantage of this aspect is that an air flow current may be created which enhances the cleaning efficiency of the system by using forced convection in addition to the potential difference to cause negatively charged particles to move towards the positive collector 200.

For example, the negative ion generator 100 may include an air moving member 110. The air moving member 110 (e.g., a fan, a Prandtl layer turbine or the like) may be used to generate an air flow 111 out of the negative ion generator 100, as exemplified in FIGS. 2, 13, 16, and 18C. The air flow 111 generated by the air moving member 110 may be used to distribute the negative ions 20 generated by the one or more ionizing sources 102 throughout the enclosed space. Distributing the negative ions 20 may improve the efficacy of the air cleaning system 10 by allowing the negative ions 20 to attach to a higher percentage of contaminants 30 at a greater distance from the ion generator 100. Additionally, distributing the negative ions 20 from a unit of air near the ionizing source 102 may reduce the generation of byproducts of dielectric breakdown. The negative current applied to a particular unit of air may more easily remain below the threshold for dielectric breakdown when the negative ions 20 are propelled by force air flow and/or convection.

Alternately, or in addition, in some embodiments, the negative ion generator 100 may include a heat source 120 (see for example FIG. 2). The heat source 120 may be powered by the power supply 104 or by a second power supply 104. The heat source 120 may be used to increase the convective efficacy of the negative ion generator 100. For example, if the air including the generated negative ions 20 is heated by the heat source 120, the air and negative ions 20 may rise in the enclosed space to a greater elevation or may rise faster.

Accordingly, one or more of the air moving member 110 or heat source 120 may be used to produce a forced air flow and/or convection in the enclosed space, thereby increasing the distribution of the negative ions 20. Increasing the distribution of the negative ions 20 may allow for more contaminants 30 to be negatively charged. In some embodiments, the forced air flow and/or convection of the negative ions 20 may be strategically designed for the specific shape and size of the enclosed space, or for enclosed spaces generally. For example, in some embodiments, the negative ion generator 100 may be positioned on the floor of the enclosed space while the collector 200 may be positioned on the ceiling of the enclosed space. The heat source 120 and/or the air moving member 110 may be used to distribute the negative ions 20 to approximately at least the head height of an occupant who is seated or standing in the room. In other words, negative ion generator 100 and collector 200 may be positioned outside the band 11 using the heat source 120 and/or air moving member 110 to move or assist in moving the negative ions 20 into the band 11. At the same time, the cross-sectional area in the horizontal plane of the air in which the negative ions are provided may be increased by the dispersion assisted by the air moving member 110 or heat source 120. Accordingly, for example, the volume of air in which the negative ions are dispersed may be in the shape of a, e.g., inverted cone. Biological contaminants are often introduced to the air by breathing, coughing, or sneezing, all of which occur at approximately head height. By increasing the number of negative ions 20 in the higher risk height near an occupant’s head (e.g., while seated or standing), a larger proportion of contaminants may be negatively charged. Similarly, by increasing the dispersion of negative ions in the horizontal plane, the uniformity of saturation of negative ions in the space and/or the surface area in the horizontal plane in which the negative ions are present may be increased thereby increasing the likelihood the droplets containing biological contaminants will be negatively charged. It will be appreciated that the potential between the collector 200 and the negative ions 20 may be set on the assumption that the negative ions 20 at least reach the head height of an occupant of the room due to forced air flow and/or convection, thereby enabling a lower potential difference to be used. Accordingly, the air moving member 110 and/or the heat source 120 may be used to move the negative ions 20 to head height, allowing the contaminants to be negatively charged, at which point they move to attach to the collector 200 on the ceiling.

In some embodiments, both of the negative ion generator 100 and the collector 200 may be positioned on the ceiling of the enclosed space. In such embodiments, the air moving member 110 may be used to move the negative ions 20 down to the head height (e.g., while seated or standing), of the user. Once the negative ions 20 negatively charge the contaminants 30, the potential from the collector 200 may cause the negatively charged contaminants 40 to loop back up and attach to the positively charged collector 200 on the ceiling, as exemplified in FIG. 11.

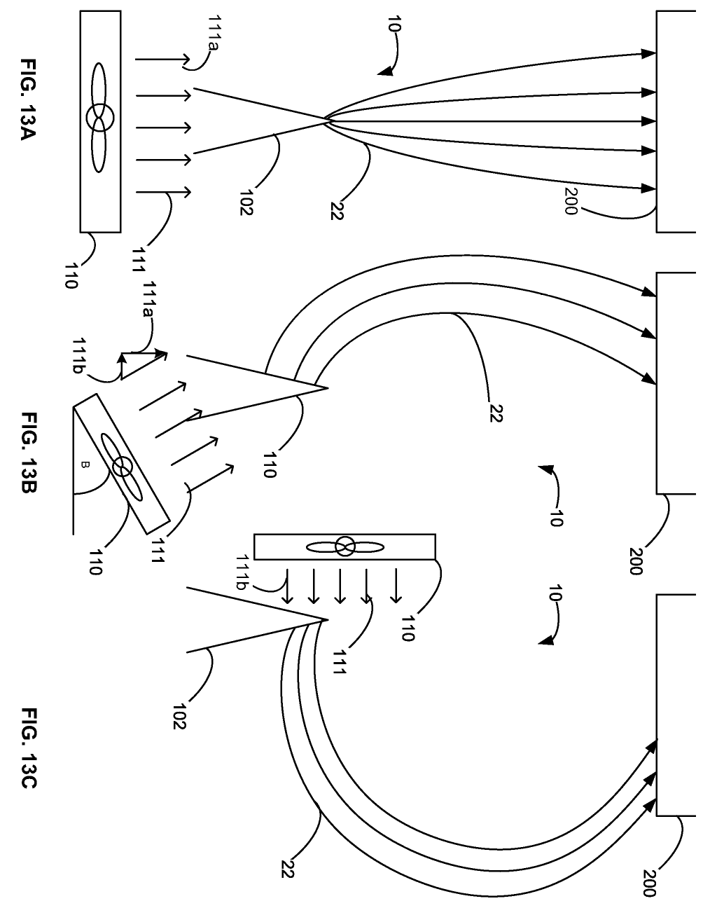

As described previously, there are various factors that may be used to vary the distribution of the electric field 22 within the enclosed space. In some embodiments, the velocity of the air flow 111 generated by the air moving member 110 and/or heat source 120 may be used to control the size and/or shape of the electric field 22. For example, each ionizing source 102 may release negative ions 20 into the air flow 111 of the air moving member 110. Depending on the velocity and angle of the air flow 111 relative to the electric field 22 (i.e. between the negative ion generator 100 and the collector 200), the shape of the electric field 22 will change. Accordingly, the use of air moving members 110 and/or heat source 120 may be used to vary the size and/or strength of the effective electric field 22. In other words, the air flow 111 may be resolvable into a first flow vector 111adirected at the collector 200, and a second flow vector 111b directed away from the collector 200, as exemplified in FIGS. 13A-13C.

For example, if the air moving member 110 is at an angle relative to the position of the negative ion generator 100 and the collector 200 and the velocity of the air flow 111 is too great, the negative ions 20 may be ejected from the electric field 22. Accordingly, the velocity of the air flow 111 may be controlled to ensure that the negative ions 20 remain within the electric field 22. In other words, the air flow 111 may have a velocity and a direction of flow selected such that the air flow 111 does not prevent the negatively charged contaminants 40 and/or negative ions 20 from travelling to the collector 200. As exemplified in FIGS. 13A-13C, the angle of the air moving member 110 relative to the electric field 22 varies the movement of the negative ions 20. As exemplified in FIG. 13A, the air moving member 110 is aligned with the electric field 22 between the negative ion generator 100 and the collector 200. Accordingly, the velocity of the air flow 111 does not significantly vary the shape of the electric field 22, since the negative ions are propelled by the air flow 111 towards the collector 200. In other words, the second air flow vector 111bis zero and the air flow 111 is directed towards the collector 200. As exemplified in FIG. 13B, the air moving member 110 is at an angle B relative to the electric field 22. Accordingly, the velocity of the air flow 111 must remain below a threshold to ensure that the propelled negative ions 20 can still be collected by the collector 200. If the velocity is too large, the ions 20 will be removed from the electric field 22.

Similarly, as exemplified in FIG. 13C, the air moving member 110 is at a 90-degree angle to the electric field 22. In other words, the first air flow vector 111a is zero. Accordingly, the velocity of the air flow 111 must remain below a lower threshold than in FIG. 13B in order to ensure the propelled negative ions 20 remain within the electric field 22. In other words, the force imparted to the air by the second flow vector 111b may be less than the field strength of the attractive force between the negative ions 20 and/or negatively charged contaminants 40, and the collector 200.

It will be appreciated that the threshold of the velocity values of the air flow 111 will vary depending on a plurality of factors, such as, but not limited to, the strength of the electric field, the shape and/or size of the enclosed space, obstacles between the air moving member 110 and the collector 200, air flow disturbances within the enclosed space, the distance between the negative ion generator and collector 200, etc. As exemplified in FIG. 13A, in some embodiments, the negative ion generator 100 may be positioned between the air moving member 110 and the collector 200, whereby the air flow 111 is at least substantially directed towards the collector 200. In some embodiments, the first flow vector 111amay include at least 50%, 60%, 70%, *0% or more of the momentum imparted to the air by the air moving member 110.

It will also be appreciated that the volume of the air flow 111 may impact the electric field 22. For example, the air flow volume may be 0.01 – 1.0 cfm per ionizer, optionally 1 – 5 cfm per ionizer, or optionally 5 – 25 cfm per ionizer.

In some embodiments, a plurality of air moving members 110 may be used to facilitate the movement of negative ions 20 within the air cleaning system 10, as exemplified in FIGS. 16 and 18C.

It will be appreciated that the position of the negative ion generator(s), the positive collector(s) 200 and/or one or more air moving member(s) 110 and/or one or more heat source(s) 120 may create an air wall to improve the removal of contaminants 30 from the enclosed space. In other words, the system may create a flow of negatively charged particles between, e.g., a customer and a store or bank clerk (see for example FIGS. 18A – 18C). The barrier of air may be used to inhibit or prevent the transition of contaminants 30 between different zones of air. Accordingly, droplets expelled by a person may be substantially or essentially conveyed to collector 200 thereby essentially creating a barrier between the customer and the store clerk It will be appreciated that the barrier of air may be used in a grocery store between the cashier and the buyer, between buyers in a queue for the cash register, between work stations in an office, between a patient and a nurses station, between a client and a receptionist and the like. Accordingly, the use of air moving members 110 may improve the safety of each occupant within the enclosed space.

It will be appreciated that the negative ion generator 100 for use in the air cleaning system 10 may be of any shape and/or size and/or the negative ion generator 100 may be provided at any location within the enclosed space. Similarly, the positively charged collector 200 may be of any shape and/or size and/or the positively charged collector 200 may be provided at any location within the enclosed space.

In some embodiments, the negative ion generator 100 and/or the positively charged collector 200 may be built into, or attached to, a light bulb or light fixture for use in the enclosed space. For example, the negative ion generator 100 may be built into a light source located on the ceiling of the enclosed space (see for example FIGS. 11 and 16). Alternately, the may be provided as part of, or attached to, a ceiling tile. As described previously, the light source may release the negative ions downwardly to the head height of an occupant before moving back to the collector 200.

In some embodiments, the negative ion generator 100 and/or the positively charged collector 200 may be adjustable. For example, if the negative ion generator 100 and/or the positively charged collector 200 is coupled to the ceiling of the enclosed space, there may be a movable rope or chain that may lower the negative ion generator 100 to a desired height. For example, if the occupants in the room are all standing, the negative ion generator 100 may be lowered to a desired height that will negatively charge a larger percentage of contaminants, such as near the occupant’s head. Similarly, if the occupants in the room are all sitting, such as in an auditorium, the negative ion generator 100 may be lowed to better charge contaminants at a seated head height. In some embodiments, the negative ion generator 100 may be height adjustable for cleaning purposes. For example, if the negative ion generator 100 is located on the ceiling, the ion generator 100 may be lowered to a height that facilitates cleaning of the ion generator 100.

In some embodiments, to better facilitate cleaning, the negative ion generator 100 and/or the positively charged collector 200 may be immersion washable. For example, the interior components of the negative ion generator 100, such as the ionizing source 102, the power supply 104, the air moving member 110, and/or the heat source 120 may be resistant to liquid such that the negative ion generator 100 may be immersed in a disinfectant for cleaning. Accordingly, for example as discussed previously, the components of the negative ion generator 100 may be sealed such that they are waterproof and/or water resistant.

In some embodiments, the negative ion generator 100 may include a removable negative electrode. The negative electrode may be removed for repair, replacement, and/or cleaning. In some embodiments, the negative ion generator 100 may include a negative electrode sensor. The negative electrode sensor may sense with then negative electrode has been removed. In some embodiments, when the negative electrode sensor detects that the negative electrode has been removed, the negative ion generator 100 may not operate.

### Portable Negative Ion Generator

In accordance with another aspect, that may be used by itself or in combination with one or more other aspects, the negative ion generator 100 may be portable. An advantage of this aspect is that the system may be used in a location wherein a fixed position negative ion generator is not readily positionable, or wherein the negative ion generator may have to be repositioned, such as if a room is reconfigurable.

For example, the negative ion generator 100 may be a wearable. For example, the negative ion generator 100 may be wearable, e.g., around a user’s neck, as exemplified in FIG. 17, or on a user’s head, as exemplified in FIG. 24 or as a broach, wrist band, name tag, lanyard and the like. The negative ions 20 may thus be released at a location near a user’s head, to improve the negative charging of biological contaminants 30 generated by the user. For example, if a user coughs or sneezes, the negative ion generator 100 may quickly negatively charge the contaminants 30 due to the close proximity of the negative ion generator 100 to the source of the contaminants.

In some embodiments, the negative ion generator 100 may be movable. For example, the negative ion generator 100 may be placed on wheels to allow for easy moving to higher risk areas. In another example, the negative ion generator 100 may be placed on a track to be movable throughout the enclosed space. For example, conference rooms may include movable partitions. Accordingly, the negative ion generator 100 may be movable to different areas of the conference room depending on the number of occupants and size of the room.

In some embodiments, the negative ion generator 100 may be autonomously moveable. For example, the negative ion generator 100 may be placed on a mobile autonomous robot, similar to a robot vacuum cleaner, a drone or the like. In some embodiments, the autonomous robot may be a vacuum cleaner. During use, the robot may move around the enclosed space, emitting negative ions generated by the negative ion generator 100 into the air. It will be appreciated that the robot may move randomly to distribute the negative ions, or may be programmed to move in a particular pattern throughout the enclosed space or optionally it may be remote controlled.

In some embodiments, the air cleaning system 10 may include one or more negative ion generators 100 fixedly mounted to a location in the physical space and one or more negative ion generators 100 that is mobile in the physical space. Accordingly, the fixed negative ion generators 100 may be strategically positioned around the physical space to generally improve the efficiency and distribution of the negative ions 20, while the mobile negative ion generators 100 may move around the physical space to further improve the efficiency and distribution of negative ions 20.

In some embodiments, a member having a mobile negative ion generator 100 may have one or more sensors to improve the distribution and efficacy of the negative ions 20. For example, the robot may have an ion sensor. The ion sensor may detect the concentration of negative ions 20 in the air of the enclosed space. The robot may then move to areas of lower negative ion concentration to improve the consistency of the negative ion distribution throughout the enclosed space. For example, regions of low negative ion concentration may indicate that contaminants have not been removed from that air space, and that portion of the air space may have a higher concentration of contaminants. A robot may use the ion sensor to determine this region of low concentration and may move there to increase the number of negative ions 20.

In some embodiments, a member having a mobile negative ion generator 100 may have a motion sensor. The motion sensor may be used to determine movement within the enclosed space. For example, once an occupant enters the enclosed space, a robot may detect motion of the occupant and may move to stay within a threshold distance of the occupant. Accordingly, the risk of biological contaminants generated by the person may be reduced very quickly before the biological contaminants spread to the rest of the enclosed space. Similarly, the robot may have a temperature sensor. The temperature sensor may be used to determine the location or presence of occupants in the enclosed space. The robot may then move to generate negative ions 20 closer to the occupant to reduce the risk of spread of biological contaminants. Similarly, an acoustic sensor may be used.

### Portable Positively Charged Collector

In accordance with this aspect, which may be used by itself or in combination with one or more other aspects, there is provided a portable positively charged collector 200 for use in the air cleaning system 10. A positively charged collector 200 may be portable in a similar manner as described previously with respect to negative ion generator 100.

It will be appreciated that the collector 200 may be positively charged in any manner that results in a net positive charge on at least a portion of the collector 200. As described previously, the value of the positive charge on the collector 200 may vary depending on the size of the room, the location of occupants, the number of the negatively charged ions and the distance between the collector and the ion generator.

### Creation of Positive Voltage

In accordance with this aspect, which may be used by itself or in combination with one or more other aspects, the positive collector 200 may be charged by a charge generator that operates at a low voltage.

Voltage is a function of current and electrical resistance. Accordingly, a large voltage may be created with a low value current and a high value resistance. Using a low current may protect occupants of the enclosed space from electrical shocks in case of inadvertent contact with the collector 200. Similarly, the use of a low current may reduce the likelihood of damage to the air cleaning system 10 itself.

It will be appreciated that the positive voltage in the collector 200 may be created and controlled by any means. For example, the collector 200 may include one or more of a Wimshurst electrostatic generator, a dirod electrostatic generator, a tribo-electrostatic generator, a Van De Graff electrostatic generator, a Tesla coil, etc. Depending on the type of positive voltage generator (e.g., a Van De Graff electrostatic generator), the speed and/or the electrode gaps may be varied to control the voltage of the collector 200. In some embodiments, the collector 200 may include a rotatable belt with a motor for rotating the belt. As the belt rotates, electrons may be moved from one side to the other, thereby creating a voltage separation across the belt.

In some embodiments, the collector 200 may include a primary coil and a planar secondary coil, also referred to as a pancake coil. The collector 200 may include a ferrite core and optionally a rectifier to control/vary the positive voltage.

In some embodiments, the collector 200 may include a ferrite core and at least one voltage doubler. The collector 200 may optionally include a rectifier.

In some embodiments, the collector 200 may include a primary coil and a multi-section secondary coil, and may include a ferrite core and a rectifier.

In some embodiments, the collector 200 may include a primary coil and a multi-sectional secondary coil, making use of a ferrite core and at least one voltage doubler. Optionally, the collector 200 may include a rectifier.

### Tesla Coil

In accordance with this aspect, which may be used by itself or in combination with one or more other aspects, there is provided a collector having a tesla coil.

In accordance with this aspect, the positive voltage in the collector 200 may be generated using a tesla coil. A tesla coil includes a primary coil and a secondary coil. The primary coil and secondary coil may operate as a transformer. That is, alternating current flow in the primary coil may generate a changing magnetic field, which may induce current flow in the secondary coil. The primary coil and secondary coil are typically configured so that the primary coil has fewer turns as compared to the secondary coil. As a result, a relatively low voltage applied to primary coil can induce a higher voltage on the secondary coil. The primary coil may be loosely coupled to the secondary coil. In other words, the primary coil may be spaced apart from the secondary coil, reducing the magnetic field of the primary coil when it is open circuited, thereby allowing the energy to remain in the secondary coil for a longer period of time.

During operation, current flow through the primary coil is interrupted by a switch, such as, but not limited to, a spark gap, transistor, or thyristor. The voltage applied across the switch is increased until a threshold voltage is reached, causing the switch to permit current to flow through the primary coil, thereby inducing current flow in the secondary coil. The switch can be configured to have a specific threshold voltage so that a particular voltage is induced on the secondary coil.

In many cases, one or more capacitors are used to increase the voltage applied across the switch. For example, a tesla coil may include a capacitor that is coupled to the primary coil, which is further coupled to a spark gap. The capacitor can be charged by a power source until the threshold voltage is reached. Once the voltage across the capacitor reaches the breakdown voltage of the spark gap, a spark is created across the spark gap, thereby allowing current to flow from the charged capacitor through the primary coil. The flow of alternating current through primary coil induces current flow in the secondary coil.

In some embodiments, as described previously, the negative ion generator 100 and the collector 200 may be located within the same system housing. The primary coil may be located at approximately the middle of the system housing. The secondary coil may extend in either or both directions from the primary coil along the length of the system housing and may be wrapped around a support tube. Accordingly, in some embodiments, the primary coil and/or the secondary coil may be tubular shaped. It will be appreciated that the support tube may be formed of a non-conductive material so as to not interfere with the operation of the collector 200. For example, the support tube may be made of glass and/or plastic.

In some embodiments, the support tube may be tapered towards one or both ends. Tapering the support tube may allow for a reduction of material in the tesla coil, without affecting the magnitude of the voltage created. The support tube may have a larger diameter in the center of the tube, near the primary coil, and a smaller diameter towards the end of the tube, resulting in a tapered secondary coil. In some embodiments, the diameter may taper such that the secondary coil forms a slightly conical shape. In some embodiments, both the primary coil and the secondary coil may taper with increasing distance from the center of the support tube.

In some embodiments, diodes may be used on either side of the primary coil to control the current flow. Accordingly, the middle portion located near the primary coil may be positively charged, forming the collector 200, and the regions outside of the diodes may be negatively charged. In some embodiments, diodes may be used to positively charge one end of the collector 200 and negatively charge the other end of the collector 200. In some embodiments, diodes may be used in a cascading orientation to stack the voltage values of the positive collector 200 and/or the negative ion generator 100.

In some embodiments, the collector 200 may include rectifiers to reduce the likelihood of sparking at the ends of the collector 200. In some embodiments, the negatively charged portions may be inductively coupled to the negative ion generator, thereby providing energizing the ionizing sources 102 in the negative ion generator 100 to release negative ions 20.

In some embodiments, the collector 200 may use flat coils, also referred to as pancake coils. For example, in some embodiments, the primary coil and/or the secondary coil may be flat pancake coils. The primary coil may be located below the secondary coil. Once current is run through the primary coil, as described above, current is induced in the secondary coil. A diode may be coupled to the tesla coil to create a negatively charged portion of the collector 200, as described above.

In some embodiments, the primary pancake coil and/or the secondary pancake coil may be tapered. Tapering the coils may reduce the material required to make the collector 200, without significantly affecting the voltage output of the coils.

### Constructing the Tesla Coil

In accordance with this aspect, which may be used by itself or in combination with one or more other aspects, there is provided various means of constructing a tesla coil for use in the collector 200.

As described previously, in some embodiments, the tesla coil may be created by winding one or more wires in the shape of a coil, spiral, or helix to form a primary coil and/or a secondary coil. The coils may be each wrapped around a support tube to produce the coil shape. Alternately or in addition, the coils may be wrapped around themselves to form the coil shape. The tesla coil may also be formed in a pancake coil, as described above.

It will be appreciated that the tesla coil may have a wire wound coil, a tapered wire wound coil, a pancake coil, a tapered pancake coil, or any combination thereof. For example, the primary coil may be wire wound while the secondary coil may be a pancake coil. In some embodiments, the primary coil may be a pancake coil while the secondary coil may be a wire wound coil. It will be appreciated that various combinations are possible.

### Conductive Ink

In accordance with this aspect, which may be used by itself or in combination with one or more other aspects, conductive ink may be used to form the primary and/or secondary coils. For example, a conductive ink may be used to form pancake coils for the primary and/or secondary coil. The conductive ink may be a liquid ink or a powder ink. It will be appreciated that any means may be used to form the coils of the conductive ink. For example, conductive liquid ink may be printed, applied by ink jet, drawn (e.g. by a liquid pen), deposited by screen printing, flowed into a groove on a substrate, applied to a heated pattern of hot melt adhesive, or any combination thereof. The conductive powder ink may be applied by a transfer member, applied by electrostatic transfer from an intermediate member, applied by a transfer member, applied to a heated pattern of hot melt adhesive, heat fused to a substrate, chemically fused to a substrate, or any combination thereof.

In some embodiments, the conductive ink may be applied to a dielectric substrate. For example, grooves may be created in the dielectric substrate in the shape of coils, and liquid conductive ink may be flowed into the groove to form the primary and/or secondary coils. In some embodiments, both thermal and chemical fusing may be used to secure the conductive ink to the dielectric substrate, thereby forming the primary and/or secondary coils. In some embodiments, the conductive ink may be mixed with a hot melt adhesive and applied in a coil pattern to the dielectric substrate. In some embodiments, the hot melt adhesive may be applied to the dielectric substrate and the conductive ink may be applied to the hot melt adhesive.

In some embodiments, the conductive ink may be used in the creation of printed circuit boards (PCBs). The PCBs may be printed with conductive ink such that primary and/or secondary coils are formed. The PCBs may then be used in the air cleaning system 10. It will be appreciated that any number of PCBs may be used. In some embodiments, two or more PCBs may be stacked on each other to save space while increasing the voltage of the air cleaning system 10. For example, a primary coil may be printed on a PCB and may be used as the base of a coil stack. A secondary coil may be printed on a PCB, and three secondary coil PCBs may be stacked on the primary coil PCB. The combined height of the coil may be, for example, ¼ inch tall. For example, each of the secondary coil PCBs may have 300 printed coils. If a 36 V AC current is applied to the primary coil PCB, the 900 secondary coils may have an output of 32,400 V. In some embodiments, an insulator may be positioned between one or more layers of the PCB primary and/or secondary coils. It will be appreciated that any number of coils may be used in the primary and/or secondary coils.

The conductive ink may include any material that is conductive and is capable of being printed, deposited, and/or transferred to form the coils. For example, the conductive ink, liquid and/or powder, may include, but is not limited to, a solvent binding and graphene nanoparticles, graphene, graphite, silver, copper, gold, nickel, tin, zinc, or any combination thereof.

In some embodiments, the conductive ink may be used in combination with the negative ion generator 100. For example, in the embodiments where the negative ion generator 100 has a conductive material to create repulsion for the negative ions 20 as discussed previously, the conductive material may be conductive ink. The negative charge may be applied from the negative ion generator 100 and may be conducted through the conductive ink throughout a substrate, such as a table mat. The table mat may then be used to repel negative ions 20 generated by the negative ion generator 100.

### Primary Coil Triggers

In accordance with this aspect, which may be used by itself or in combination with one or more other aspects, there is provided a collector having various means of triggering the tesla coil. The primary coil trigger may be controlled by any means capable of varying the current in the tesla coil. For example, the air cleaning system 10 may have one or more sensors that detects one or more of the electric field strength, airborne pollutant concentration, noise in the enclosed space, number of negative ion generators, number of occupants in the enclosed space, motion, thermal signatures, or any combination thereof. The control system of the air cleaning system 10, including the various sensors and detectors will be discussed in more detail subsequently.

The trigger of a tesla coil provides the initial current transfer that creates an AC current in the transformer. For example, as described above, the tesla coil may have a spark excited primary coil. Once a threshold voltage is reached, the spark excited primary coil generates a spark across the spark gap, thereby completing the primary circuit and allowing current to flow through the primary coil. The size of the spark gap in the primary circuit may be varied depending on the desired use of the primary circuit. For example, if a higher positive voltage is required, the spark gap size may be increased. Increasing the spark gap results in a higher threshold voltage to complete the primary circuit and corresponding current to flow through the primary coil. Once the spark arcs across the spark gap, current flows through the primary coil, inducing current in the secondary coil, and generating the voltage output of the secondary circuit.

It will be appreciated that the spark gap size may be constant, or may be varied to control the voltage of the collector 200. For example, if the number of occupants in the room increases, the risk of contamination increases. Accordingly, one or more sensors may determine that an increase in voltage is required to ensure the safety of the occupants in the room. Accordingly, one or more sensors may issue a signal to an actuator that adjusts the spark gap. The actuator may be any electromechanical or thermomechanical member that may adjust the gap size based on an input from the sensors. The spark gap may be coupled to, for example, gears that may be controlled by a motor in the collector 200 (an electromechanical actuator). The gears may increase and/or decrease the spark gap size to control the voltage output. In some embodiments, a solenoid, gears, and/or a voice coil may be used to control the size of the spark gap.

It will be appreciated that any style of trigger may be used in the collector 200. For example, the trigger may include, but is not limited to, a spark excited primary, a static triggered spark gap, a rotary spark gap, a single resonant solid state trigger, a dual resonant solid state trigger, a triple resonant solid state trigger, a singing (musical) coil, a continuous wave trigger, a double resonant trigger, a triple resonant trigger, or any combination thereof.

In some embodiments, backup primary coils and/or secondary coils may be used in the collector 200. For example, if the maximum voltage threshold is reached for a current system, and the air cleaning system 10 detects that an increased voltage is required, additional primary coils and/or secondary coils may be coupled with the primary and/or secondary circuit. Increasing the number of coils may increase the voltage output of the collector 200. For example, if the air cleaning system 10 determines that there are more occupants than usual, or more contamination than usual, the backup coils may come online to increasing the ionizing power of the air cleaning system 10. In a similar manner, a system (which may or may not use any tesla coils) may have multiple collectors 200 and one or more additional collectors 200 may be brought on line to increase the potential difference.

For example, in some embodiments, the collector 200 may have primary coils (first stage), secondary coils (second stage), and a third stage. The third stage of the collector 200 may act as the voltage output of the tesla coil. In some embodiments, the third stage may include a rectifier. The secondary coils may energize the third stage, thereby providing a positive voltage to the collector 200.

In some embodiments, for example when the primary trigger is a singing coil, the cover 210 of the collector 200 may provide acoustic resonance matching.

### Structure and/or Position of Positive Collector

In accordance with this aspect, which may be used by itself or in combination with one or more other aspects, an air cleaning system has one or more collectors which may be of various designs and may be at various locations wherein one or more of the collectors may be moveably mounted.

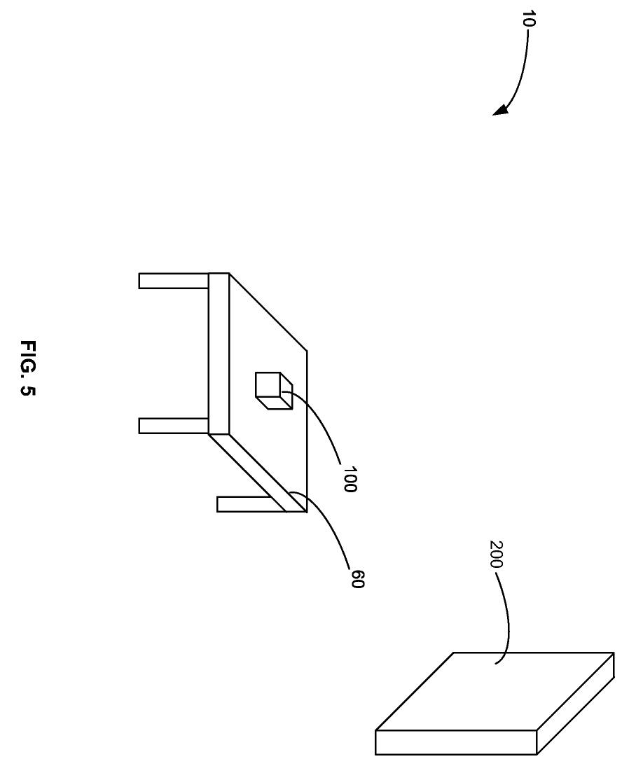

It will be appreciated that the collector 200 may be any shape or size capable of collecting negatively charged contaminants 40. For example, in some embodiments, the collector 200 may be one or more point sources located throughout the enclosed space. The point source collector 200 may be located anywhere in the enclosed space, preferably on the wall or ceiling. For example, depending on the height of the enclosed space, to decrease the required potential, the collector 200 may be located on the wall of the enclosed space, as exemplified in FIGS. 5-8. It will be appreciated that if a system uses multiple collectors, then the collectors 200 may be the same of different.

In some embodiments, to improve the collection of negatively charged contaminants 40, the surface area of the collector 200 may be increased. For example, the collector 200 may be tubular, round, oval, triangular, square, rectangular, or any combination thereof. It will be appreciated that, as described previously, the collector 200 may be curved such that the edges of the collector 200 have a radius such that the production of ozone and/or cations is reduced and/or eliminated.

As described previously, the positive housing of the collector 200 may be fully enclosed to reduce the likelihood of dielectric breakdown byproducts from being released into the enclosed space. For example, the collector 200 may be any enclosed tubular shape. As described previously, having a rounded housing for the collector 200 may reduce the likelihood of dielectric breakdown outside of the housing. For example, the collector 200 may be toroidal.

Accordingly, the tubular housing of the collector 200 may form any shape. For example, the tubular housing may meander along the ceiling of the enclosed space. The tubular housing may be positioned along the ceiling such that the lighting and/or decoration is not affected by the collector 200.

In some embodiments, the shape of the tubular housing may be decorative, to accentuate the existing decorations in the enclosed space and/or to act as its own decoration. In some embodiments, the shape of the collector 200 may form a logo and/or symbol, such as a corporate logo.

In some embodiments, the collector 200 may be substantially flat. For example, the collector 200 may be rectangular. For example, the collector 200 may be a panel, as exemplified in FIG. 1. As exemplified, the collector 200 is located on the ceiling of the enclosed space. In some embodiments, the collector 200 may be built into the ceiling as a ceiling tile. It will be appreciated that the rectangular collector 200 may have radiused edges to reduce the production of ozone and/or cations, as described previously.

In some embodiments, the collector 200 may be included in a light source on the ceiling or wall of the enclosed space, as exemplified in FIGS. 5 and 11. For example, a light bulb may include a positive electrode 202 that is positively charged to form the collector 200. As exemplified, the positive electrode 202 may be a metal plate. In some embodiments, the collector 200 may be both a ceiling tile and a light source. In some embodiments, the collector 200 may be the cover of a light source such as a fluorescent light.

In some embodiments, there may be a plurality of collectors 200. For example, the ceiling of the enclosed space may have a plurality of ceiling tile collectors 200. In some embodiments, the collector 200 may form the entire ceiling of the enclosed space. In some embodiments, the plurality of collectors 200 may surround an element of the enclosed space. For example, a toroidal collector 200 may surround each light source in the room.

In some embodiments, the collector 200 may be shaped to accent a feature of the enclosed space. For example, it may be a frame for a poster or other work of art, or a light fixture such as a fluorescent light fixture or a window frame.

In some embodiments, the collector 200 may be strategically shaped to improve the collection of negatively charged contaminants 40 in the enclosed space. For example, the collector may be extended around the perimeter of the area in which people are expected to be when in the space. For example, if the space is a conference room having a conference table that is rectangular, then the collector 200 may be tubular, may extend in the shape of a rectangle, and may be positioned over a rectangular table (e.g., the shape may be the same as the perimeter of the table and may have the same dimension as the table, or it may be larger to be positioned outward of the table or it may be smaller to be slightly within the perimeter of the table). Accordingly, the shape of the collector 200 may be designed to accommodate the region in the enclosed space that is most likely to have contaminants e.g., over the table. It will be appreciated that the collector 200 may be shaped to accentuate any aspect of the enclosed space, such as, but not limited to, a dining table, gaming table, pool table, slot machine, arcade games, a couch, etc. Similarly, the collector 200 may be shaped to avoid aspects of the enclosed space, such as avoiding a staircase to prevent the attraction of contaminants 40 to heights where an occupant may frequently stand/walk. It will be appreciated that if a collector 200 is a tubular element that extends around an area, then an additional collector may be positioned, e.g., inwardly of the area framed by the tubular collector 200.

As described previously, the collector 200 may be positioned to hang horizontally, such as along a ceiling. Such a collector may be a light fixture that is mounted in a ceiling or suspended from the ceiling. It may also be incorporated into any element that is mounted to a ceiling. It will be appreciated that, if the collector 200 is mounted to a ceiling, then the height of the collector 200 may be adjustable (manually or by a motor). For example, as described previously for the negative ion generator 100, the collector 200 may be lowered from the ceiling of the enclosed space. By lowering the collector 200, the desired potential difference may be lowered to conserve energy, since the collector 200 is located closer to the occupants of the room. Similarly, once the collector 200 has collected a threshold amount of negatively charged contaminants 40, the collector 200 may be lowered and more easily disinfected. In some embodiments, the height of the collector 200 may be adjusted to improve the efficacy of the air cleaning system 10. For example, depending on the use of the enclosed space, the collector 200 may be lowered to the optimal height for removing contaminants 30 from the air. For example, the collector 200 may be lowered when, e.g., more people are in a space and more contaminants may be present.

Alternately, or in addition, the collector 200 may be moveable along the ceiling (along the length or width of the space) so as to overlie a different part in the space, e.g., to areas which are expected to have a higher concentration of contaminants, such as when people move to different regions of the space. If multiple collectors 200 are provided, then the location of one or more of the collectors 200 above the floor of the space may be adjusted to provide more collectors 200 at a location that has more people located therein. The position of the collectors 200 may be manually adjustable, remotely controlled by a person or automatically self-adjusting based on an input signal from one or more sensors as discussed herein.

For example, the collector 200 may be positioned on a track on the ceiling of the enclosed space. The air cleaning system 10 may include one or more detectors, which will be discussed in more detail subsequently, for controlling the position of the collector 200. For example, the air cleaning system 10 may have an ion detector. The ion detector may be used to determine the concentration of negatively charged contaminants 40 in different regions within the enclosed space. The collector 200 may be moved, manually adjustable, remotely controlled by a person or automatically self-adjusting based on an input signal from one or more sensors as discussed herein, to the regions of higher contaminate concentration, to improve the contaminate removal efficiency.

As described previously, the collector 200 may be positioned to hang vertically, such as along a wall or extending downwardly from the ceiling. In such a case, collector may be adjustable in a similar manner as discussed previously with a collector 200 that is horizontally mounted to a ceiling. For example, if the space is a sports venue, then the collector(s) 200 may be incorporated into a team banner or the like which is suspended from the ceiling or above the seating area. The banners may be lowered when people are seated to increase the driving force of the electric field.

In some embodiments, the collector 200 may be supported by a base such that the collector 200 may extend upwardly from the floor or table. In such a case, the collector(s) 200 may also be height adjustable as discussed herein.

In some embodiments, the air cleaning system 10 may include a ground collector. For example, one or more collectors 200 may be positioned on the floor of the enclosed space. The ground collector 200 may operate at the same or different voltage as a ceiling or wall positioned collector 200. For example, if both a ground collector(s) 200 and a ceiling collector(s) 200 are provided, then in some embodiments, the ground collector(s) 200 may operate at a lower voltage than the ceiling or wall positioned collector(s) 200. Operating at a lower voltage may allow the majority of negatively charged contaminants 40 to move to the higher areas in the enclosed space, thereby improving the safety of the occupants. For example, the ceiling and/or wall positioned collector(s) may have a voltage that optimizes the removal of contaminants 30 from the average head-height of an occupant. The ground collector may collect negatively charged contaminants 40 that are below the optimized height for the other collector(s). Collecting the excess contaminants may reduce the likelihood of infection/contamination of occupants. Alternately, or in addition, the ground collector(s) may be used to secure the fallen negatively charged contaminants 40 such that walking does not disturb the contaminants back into the air. It will be appreciated that, alternately, only a ground collector(s) may be provided.

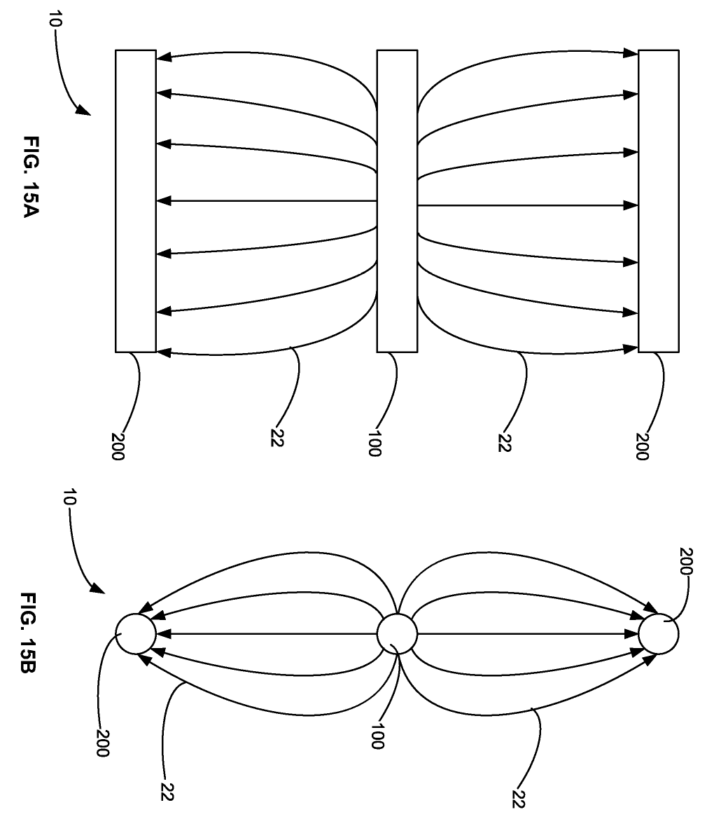

For example, the air cleaning system 10 may include a ceiling collector and a ground collector, as exemplified in FIGS. 15A and 15B. As shown, the negative ion generator 100 is located between the ground collector 200 and the ceiling collector 200. The released negative ions 20 may charge contaminants 30 before moving to the closer of the two collectors 200. Accordingly, the air cleaning system 10 may be used for enclosed spaces where the head height of the occupant changes. For example, if occupants of the enclosed space move from standing to sitting, the air cleaning system 10 may capture contaminants at both heights.

In some embodiments, the ground collector may be neutrally charged. A neutral ground collector may be used to collect and/or secure fallen contaminants 40, without interfering with the wall and/or ceiling positioned collectors that are designed to remove contaminants 40 from the average head-height of occupants.

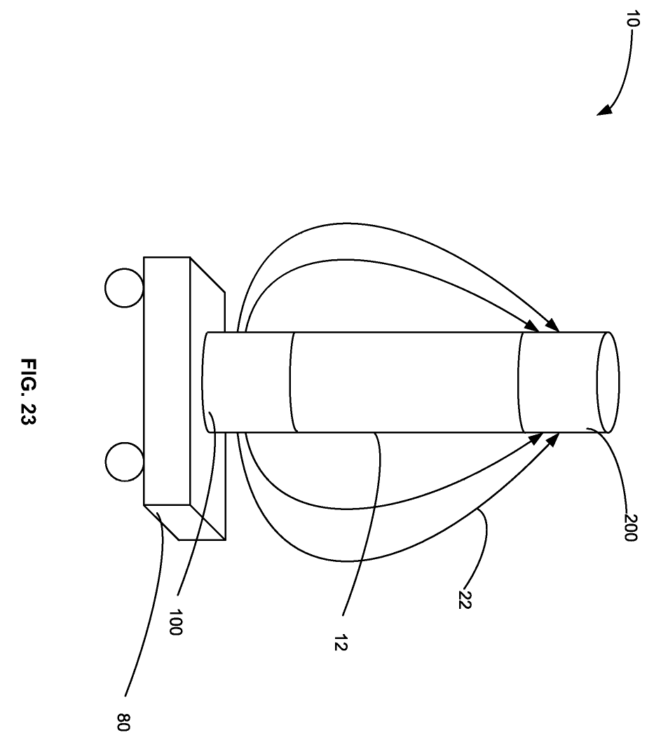

In some embodiments, the collector 200 may be movable even if not mounted to a wall or ceiling. For example, the collector may be provided on a mobile platform such as an autonomous robot as exemplified in FIG. 23 and/or in drones. It will be appreciated that a movable collector 200 may be manually moveable (e.g., it may be on a wheeled base as exemplified in FIG. 23 and it may be pushed into a desired position by a person). Alternately, or in addition, the movable collector 200 may be motorized and may be remotely controlled by a person. For example, a person may be able to use a user interface (such as a smart phone) to direct a movable collector 200 to an alternate location. If a plurality of movable collectors 200 are provided (e.g., in a convention hall or display space), then an operator is a control booth may be able to control, optionally individually, the position of each movable collector 200. Alternately, or in addition, movable collector 200 may be automatically movable. As discussed previously, one or more sensors may provide an input signal to, e.g., a controller that may be part of a movable collector 200 and the controller may provide drive signals to one or more motors of the movable collector 200.

### Cleaning/Disinfecting Collector

In accordance with this aspect, which may be used by itself or in combination with one or more other aspects, there is provided a collector that is cleanable and/or replaceable.

As the collector 200 operates in the air cleaning system 10, the collector 200 will collect many contaminants 40. Optionally, the collector 200 may be disposable or cleanable. Alternately, or in addition, in some embodiments, the collector 200 may be covered or coated to allow for disposal or sanitization. For example, the collector electrode 202 may be a metal plate that is conductive to allow the collector 200 to be positively charged. Rather than cleaning the collector 200 by wiping it with a disinfectant, the collector 200 may have a cover 210 positioned over the metal plate 202 (see for example FIG. 14C). For example, the cover may be made of paper or cloth. After a certain period of time, the cover 210 on the collector 200 may be removed and disposed of to get rid of any collected contaminants. A new cover 210 may then be placed over the collector 200 for its next use. It will be appreciated that the disposable cover 210 may be formed of any disposable, biodegradable, and/or recyclable material, including, but not limited to, paper, cardboard, biodegradable plastic, plastic, cloth, or any combination thereof.

In some embodiments, the cover 210 for the collector 200 may be made of an electrical insulating material to cover the conductive metal plate 202 of the collector 200. Insulating the conductive collector 200 may reduce the risk of shock to an occupant in the enclosed space. As described previously, the risk of shock may be low when the high voltage of the collector 200 is generated by a large resistance, rather than a large current.

In some embodiments, the collector 200 may be decorative. Accordingly, in some embodiments, the cover 210 for the collector 200 may be decorative. Accordingly, the air cleaning system 10 may be used without affecting the aesthetic of the enclosed space. In some embodiments, the collector 200 may be covered with art, transparent, or may be designed to be aesthetic. For example, in some embodiments, the collector 200 may include an indium tin oxide electrode with a transparent polycarbonate outer insulation layer. Accordingly, the collector 200 may be at least partially transparent. Having an at least partially transparent collector 200 may allow for the build-up of contaminants 40 on the surface of the collector 200 to more easily be seen, thereby providing a better indication as to when the collector 200 should be cleaned.

In some embodiments, the collector 200 may be raised or lowered for cleaning.

### Self-Sanitizing and /or Self-Cleaning Collector

In accordance with this aspect, which may be used by itself or in combination with one or more other aspects, there is provided a positively charged collector 200 that is self-sanitizing and/or self-cleaning. Having a self-sanitizing collector 200 may reduce the risk of infection caused by a person cleaning a contaminated surface. Accordingly, the collector 200 may operate without needing any contact by a person.

In some embodiments, the collector 200 may be made of or may include a material that kills and/or denatures biological contaminants, such as viruses, also referred to as a self-disinfecting member. The self-disinfecting member may treat the collector 200, such as an outer surface of the collector 200. For example, the self-disinfecting member may include, but is not limited to, indium tin oxide, copper, or silver. Accordingly, the collector 200 may be coated with an anti-bacterial or anti-viral coating to reduce the build-up of live bacteria and/or viruses on the collector 200.

Alternately, or in addition, the cover of the collector 200 may be made of a material that kills or denatures contaminants. In some embodiments, the cover 210 of the collector 200 may be capable of wicking a disinfectant material over the collector 200 and/or cover 210. For example, the collector 200 may include a reservoir of disinfectant. The cover 210 may be in contact with the reservoir such that disinfectant wicks through the cover 210 and contacts the collector 200. Once the reservoir has been depleted, the disinfectant may be replenished without ever requiring a person to contact the collector 200 itself.

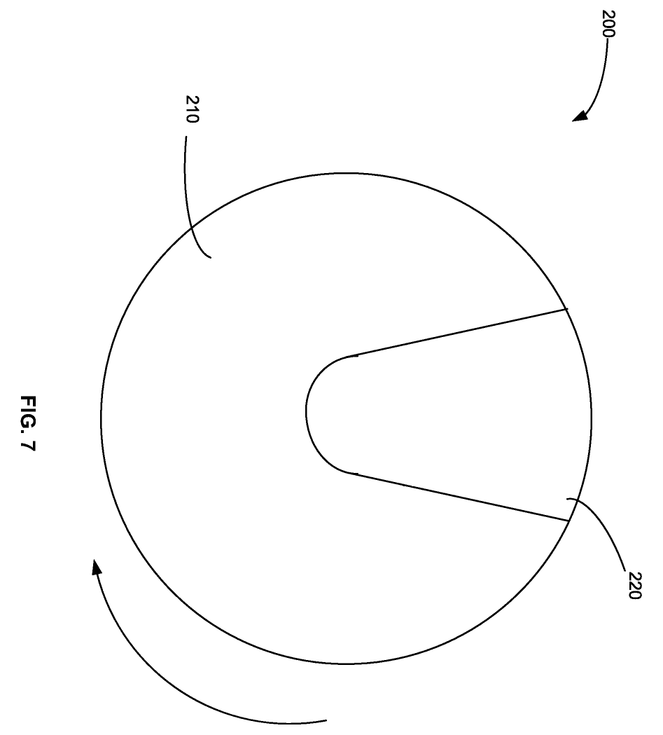

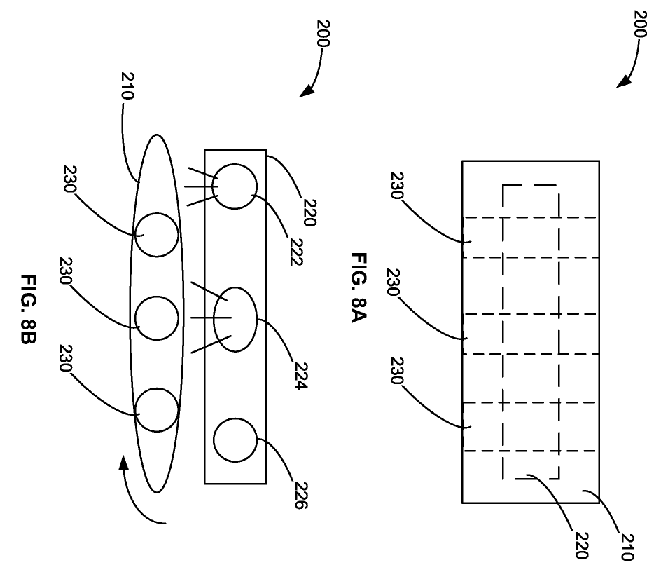

In some embodiments, the collector 200 and/or cover 210 may be moveable (e.g., rotatable) with respect to a disinfecting module 220 to disinfect the collector 200 and/or cover 210. For example, the disinfecting module 220 may be stationary and the collector 200 and/or cover 210 may be moveably mounted to move the collector 200 and/or cover 210 to the disinfecting module 220. Alternately, the disinfecting module 220 may be moveable to traverse along the collector 200 and/or cover 210. For example, the collector 200 may be wheel-shaped, as exemplified in FIG. 7, and may rotate in the direction indicated by the arrow. As exemplified, the wheel-shaped collector 200 may have a disinfecting module 220 that covers at least a portion of the collector 200 and/or cover 210 if a cover 210 is provided, for disinfecting the collector 200 and/or cover 210. Alternately, the collector 200 and/ or the cover 210, may be rotatable about one or more rolls 230, as exemplified in FIGS. 8A and 8B. As the collector 200 rotates, the portion that is disinfected changes with the rotation. For example, the collector 200 may rotate at speed sufficient to disinfect the covered portion under the disinfecting module 220. The disinfecting module 220 may make use of any material or technology capable of disinfecting the collector 200. For example, the disinfecting module 220 may use UV light, disinfectant spray, disinfectant liquid, high heat, etc. The rotation speed of the collector 200 may vary according to the type of disinfecting module 220. As exemplified in FIGS. 8A and 8B, the disinfecting module may include both a disinfectant spray 222 and a UV light 224. It will be appreciated that the positive charge of the collector 200 may be applied to any component of the collector 200. For example, the positive charge may be applied to one or more of the metal plate 202, the cover 210, the rollers 230, or the disinfecting module 220.

In some embodiments, as exemplified in FIGS. 8A and 8B, the disinfecting module 220 may include a contaminate sensor 226. The contaminate sensor may be used to determine the level of contamination of the collector 200. Once the level of contamination on the collector 200 reaches a threshold value, the collector 200 may be rotated to disinfect the portion of the collector under the disinfecting module 220. The level of contamination of the collector 200 may be determined by various methods. For example, the contamination sensor may be an optical sensor that scans the collector 200 for contaminants. In some embodiments, the contaminate sensor may be used to determine the occupancy of the enclosed space, and may calculate the statistical level of contamination for that occupancy level. For example, the contaminate sensor may be an acoustic sensor or a motion sensor, a counter provided at an entrance to the enclosed pace, or the like. In some embodiments, the contaminate sensor may determine the occupancy of the room by detecting the number of cell phones present. Accordingly, once the level of likely contamination is determined, the collector 200 may vary its rotational speed and/or rate of cleaning the collector 200. Alternately, or in addition, the disinfecting module 220 may be actuated on an intermitted basis based on, e.g., a schedule.

It will be appreciated that the collector and/or the disinfecting module 220 may be otherwise moveable (e.g., translatable).

Alternately, or in addition, the temperature of the collector 200 and/or the cover 210 may be raised to denature a biological contaminant thereon. The temperature may be intermittently raised or the collector 200 and/or the cover 210 may be at the elevated temperature at all times that the system 10 is in use.

For example, the collector 200 may have a heat source that heats the collector 200 and/or cover 210 if a cover 210 is provided to an acceptable temperature. It will be appreciated that the temperature of the collector 200 may be varied depending on the desired use of the air cleaning system 10 and the desired speed of decontamination. For example, a biological virus, such as COVID-19, may be denatured within 20 seconds at a temperature of 80°C. However, COVID-19 may be denatured in approximately one minute at 60°C. In some embodiments, the collector 200 may be intermittently heated to conserve energy. For example, the contaminate sensor 226 may determine when a threshold level of contaminate has been collected by the collector 200, and may initiate the heat source to sanitize the collector 200.

In some embodiments, the heat used to kill/denature contaminants 40 may be inherent in the design of the collector 200. For example, as described above, the collector 200 may be a part of a light source such that the collector 200 is thermally coupled to the light source and is therefore heated by the light source. In some embodiments, the light source may be incandescent light, capable of reaching upwards of 120°C, a sodium light or the like. Accordingly, when the light source is turned on, the collector 200 may be disinfected.

In some embodiments, the collector 200 may include an outer layer that is thin and subjectable to high heat. For example, the positive housing 16 may include a thin outer layer (e.g. 0.0001” thick) that is connected to a heat source. During use, the heat source may be used to rapidly heat the thin outer layer to high temperatures for a brief period of time. For example, the thin outer lay may be heated for a few seconds between 80-200°C or optionally 100-150°C. The high heat may be hot enough to denature a biological contaminate 30, while the thin outer layer is thin enough that there is insufficient thermal mass to burn an occupant within the enclosed space. Additionally, the insufficient thermal mass of the thing outer layer prevents the thin outer layer from melting the substrate (e.g., plastic) on which it is deposited. It will be appreciated that the thin outer layer may be made of any one or more materials capable of being subjected to relatively high heats. For example, the thin outer layer may be made of, including, but not limited to, nickel, aluminum, copper, zinc oxide, tin oxide, aluminum oxide, or any combination thereof. Accordingly, the thin outer layer may be used to rapidly denature and/or kill contaminants 30 collected by the collector 200 without harming the occupant. The thin outer layer may be cyclically heated to ensure that collected contaminants 30 are denatured and/or killed frequently.

It will be appreciated that the thin outer layer may be formed by any means. For example, the thin outer layer may be formed by, including but not limited to, vapour deposition such as PVD and/or CVD, sputter, or any combination thereof.

### Controlling the System Operation

In accordance with this aspect, which may be used by itself or in combination with one or more other aspects, there is provided an air cleaning system with a means for controlling the operation of the air cleaning system. Once the status of the system operation has been determined, the system may be controlled, manually, automatically, and/or remotely, to improve the operation of the air cleaning system.

For example, in some embodiments, as described previously, the air cleaning system 10 may include detectors and/or sensors, together referred to as detectors, for use in controlling the system. The detector(s) may be used to detect, for example, the electric field strength, the level of airborne contaminants, the concentration of contaminants , the noise level, the number of negative ion generators available or in operation, the number of occupants in the enclosed space (e.g. by motion sensors, thermal sensors, cameras, entry and exit monitoring, a wearable which is detectable by a detector or some combination thereof), the position of the occupants in the enclosed space, the level of negative ions, radio frequencies, or any combination thereof.

It will be appreciated that the detectors may be positioned anywhere inside or outside the enclosed space. For example, the detectors may be positioned within the enclosed space on the floor, wall, ceiling, table, etc. The detectors may be positioned within the wall, ceiling, table, floor, etc. The detectors may be positioned on or within one or more of the negative ion generator 100 or the collector 200.

Accordingly, the air cleaning system 10 may include, e.g., a controller, that receives input from the detector(s) and provides a signal indicative of an operational state of the system 10. Alternately, or in addition, the detector(s) themselves may issue the signal. For example, a signal may be provided indicating that the negative ion concentration is too low, too high, or within a sufficient range. Similarly, a signal may be provided indicating that the collector 200 is operating at too low of a voltage, too high of a voltage, or within a sufficient range. In some embodiments, the signal may be an audio signal, such as an alarm or beeping, a visual signal, such as a light, or both. For example, an ionization detector may be positioned within the negative ion generator 100 to measure the output of the negative ions. An LED may be positioned on the exterior of the negative ion generator 100, and may indicate the status of the ionization detector. For example, the LED may be lit green if a sufficient rate of negative ions 20 are being produced, and may be lit red if there is an insufficient rate of negative ions 20 being produced. Similarly, an RF detector may be positioned within the collector 200, and may use visual and/or audible signals to indicate the operation status of the device. For example, if the collector 200 fails (e.g. lacks a sufficient voltage), an alarm may go off to warn the occupants of the room that the contaminants are no longer being removed at the same rate.

If the air cleaning system 10 includes a processor and/or a controller, then the detectors in the air cleaning system may communicate with the processor and/or controller by any communication means, including, but not limited to, radio frequency, Bluetooth, infrared, a wired connection, Wi-Fi, NFC, or any combination thereof. For example, the detectors may send data to the processor and/or controller regarding the status and operating levels of one or more of the negative ion generator 100 or the collector 200.

In some embodiments, the air cleaning system 10 may be remotely controlled. Accordingly, a user may alter one or more operating parameters of the system in response to a signal(s) issued by a detector(s). For example, a user may control the rate of negative ion generation and/or the voltage value of the collector 200 in response to a warning signal issued by a detector(s). Controlling the negative ion generation and/or the positive voltage may allow a user to vary the strength of the electrical field in the enclosed space, thereby altering the rate of contaminate removal. For example, if more occupants enter the room, the rate of contaminants may increase. Accordingly, the user may remotely increase the strength of the electric field to compensate for the increase in contamination. In some embodiments, the air cleaning system 10 may be connected to the Internet, to allow for remote status access and/or remote control of the air cleaning system 10. It will be appreciated that a user may manually adjust one or more operating parameters of the system even if a warning signal is not issued.

Alternately, or in addition, the controller may adjust one or more operating parameters of the system in response to a signal issued by a detector(s) and/or a pre-programmed schedule. For example, the air cleaning system 10 may have a control feedback loop. The controller in the air cleaning system 10 may automatically adjust the rate of negative ion generation and/or the strength of the positive voltage in the collector 200 to compensate for an increase or decrease in contaminants 30 in the enclosed space. For example, as described previously, the voltage of the collector 200 may be varied by changing the current through the collector 200 and/or by varying the switch sensitivity of the collector 200. Similarly, the negative voltage of the negative ion generator 100 may be increased to increase the rate of negative ion 20 generation.

In some embodiments, the voltage and current in the air cleaning system 10 may be measured to monitor the operation of the system 10. For example, the current input into the negative ion generator 100 may be measured along with the voltage output. If the voltage output begins to decrease with the same current input, the system 10 may recognize that the negative electrode 102 may need to be replaced.

In some embodiments, as described previously, the position of the negative ion generator 100 and/or collector 200 may be altered to compensate for an increase or decrease of contaminants in one or more areas of the enclosed space. For example, if the air cleaning system 10 detects an increase in the number of occupants in the room, the height of the collector 200 may be decreased to increase the strength of the electric field. In some embodiments, the negative ion generator 100 may move to regions of higher contamination and/or higher occupancy to increase the rate of negatively charging the contaminants 30.

In some embodiments, as described previously, the voltage strength of the negative ion generator 100 and/or collector 200 may be varied depending on the distance between them. For example, as exemplified in FIG. 17, the negative ion generators 100 are worn by occupants in the enclosed space. Accordingly, as an occupant moves further away from the collector 200, the distance between the negative ion generator 100 and the collector 200 increases. To ensure that the field strength remains at a sufficient level to remove contaminants despite the increased distance, the field strength may be increased by increasing the voltage of the negative ion generator 100 and/or the collector 200. In embodiments where the negative ion generators 100 are powered by a battery, the variation in field strength due to distance may be used to conserve battery life. For example, the collector 200 may remain at a constant voltage, but as the negative ion generator 100 moves closer to the collector 200, the negative voltage may automatically be decreased to conserve energy. Accordingly, the operating lifespan of the negative ion generator 100 may be increased without increasing the risk of contamination to the occupant. In some embodiments, the field strength may remain generally constant as the location of a person in the physical space varies. For example, as a person moves further from the negative ion generator 100, the voltage of the negative ion generator 100 may be increased and/or the negative ion generator 100 may move closer to the person. Alternately, or in addition, the voltage of the positive collector 200 may be increased and/or the positive collector 200 may move closer to the person.

In some embodiments, the air cleaning system 10 may provide a signal indicating that a threshold level of contaminants 40 has been collected by the collector 200, signaling that the collector 200 should be cleaned.

In some embodiments, the detectors may detect the presence of a cleaning action. For example, the detectors may detect the presence of a solvent cleaner on the negative ion generator 100 or the collector 200, the use of a mechanical cleaning element, the use of a liquid cleaner, the removal of the cover 210 of the collector 200 and/or the subsequent replacement of the cover 210, the use of a UV light or other electromagnetic energy, or any combination thereof. In other words, the air cleaning system 10 may detect when the negative ion generator 100 and/or the collector 200 has been cleaned. Tracking the cleaning of the components of the air cleaning system 10 may provide a base level of sanitization for the system to recognize when it should be cleaned again. For example, in some embodiments, the air cleaning system 10 may measure the voltage of the collector 200 when the collector 200 has been cleaned. As the air cleaning system 10 is used, the negatively charged contaminants 40 build up as debris on the surface of the collector 200. A build-up of debris may result in a decrease in current and/or voltage through the collector 200, indicating that the collector 200 needs to be cleaned. Once a threshold value is reached, the air cleaning system 10 may provide an alert that the collector 200 needs to be cleaned, or may initiate an automatic cleaning of the collector 200, as described previously.

In some embodiments, the air cleaning system 10 may include a clock circuit (e.g., timer). The clock circuit may be used to track the cleaning of the collector 200 and/or the negative ion generator 100. For example, once the collector 200 has been cleaned, the clock circuit may be restarted. The clock circuit may then send a signal after a period of time to indicate that the collector 200 should be cleaned again. In some embodiments, the clock circuit signal may be sent after a certain loading value has been reached. For example, the signal may be sent after a particular loading reaches a certain threshold, including but not limited to, the debris build-up on the collector 200, the number of occupants in the room, the level of negative ions in the air, or any combination thereof.

### Increased Voltage Mode

In accordance with this aspect, which may be used by itself or in combination with one or more other aspects, there is provided an air cleaning system with a mode of increased voltage. As described above, increasing the negative voltage of the negative ion generator 100 may result in the formation of ozone. However, in some situations, such as a chemical accident or fire, the generation of ozone may be a lower risk than allowing the contaminants to remain in the enclosed space. Accordingly, in some embodiments, the air cleaning system may have a boost mode where the negative ion generator 100 and/or collector 200 operates at a much higher voltage level.

For example, in the case of a fire, the negative ion generator 100 may increase in voltage to maximize the output of negative ions 20, without concern for the ozone levels. Increasing the rate of negative ion 20 productive may increase the speed of contaminate removal from the enclosed space. Accordingly, the smoke in the enclosed space may be removed very rapidly. The generated ozone in the enclosed space may dissipate after a few hours. In other words, the boost mode may be used in situations where removing the contaminants rapidly is favoured over allowing occupants back into the room.

In some embodiments, the air cleaning system 10 may include an ozone destructor. An ozone destructor breaks down ozone in the air. Accordingly, when the boost mode is enabled, the ozone destructor may help reduce the ozone in the enclosed space.

### Exemplary Air Cleaning Systems

The following is a discussion of exemplary systems for a space. Each example utilizes various elements discussed previously. It will be appreciated that each example is exemplary and each system discussed may use additional elements disclosed previously or optionally fewer elements.

As described previously, it will be appreciated that the air cleaning system 10 may be used in any enclosed, or partially enclosed, space. The following is a list of possible uses of the air cleaning system 10, including, but not limited to: conference rooms, dental offices, churches, restaurants, schools, movie theatres, auditoriums, office buildings, transportation vehicles (e.g. cars, trains, busses, airplanes, ambulances, subways), residential homes, apartment buildings, voting booths, government buildings, casinos, universities, retail stores, doctor’s offices, hospitals, meeting rooms, hotels, motels, amusement parks, ships, waiting rooms, hallways, radiation rooms, drones, vacuums, pet beds, masks, personal pendants, etc.

The components and/or position of components in the air cleaning system 10 may vary depending on its desired use. For example, in some embodiments, the air cleaning system 10 may have a plurality of negative ion generators 100 and a single collector 200, as exemplified in FIGS. 9, 11, 16, 17, 19, and 20. In such embodiments, the collector 200 may be sized to collect negatively charged contaminants 40 from the plurality of negative ion generators 100.

Depending on the size and shape of the room, the position of the collector 200 may vary. For example, as exemplified in FIGS. 5-8, the collector 200 is located on the wall of the enclosed space. Positioning the collector 200 on the wall may reduce the energy required to maintain a sufficient potential difference between the negatively charged contaminants 40 and the collector 200, since the collector 200 is at a lower elevation than the ceiling.

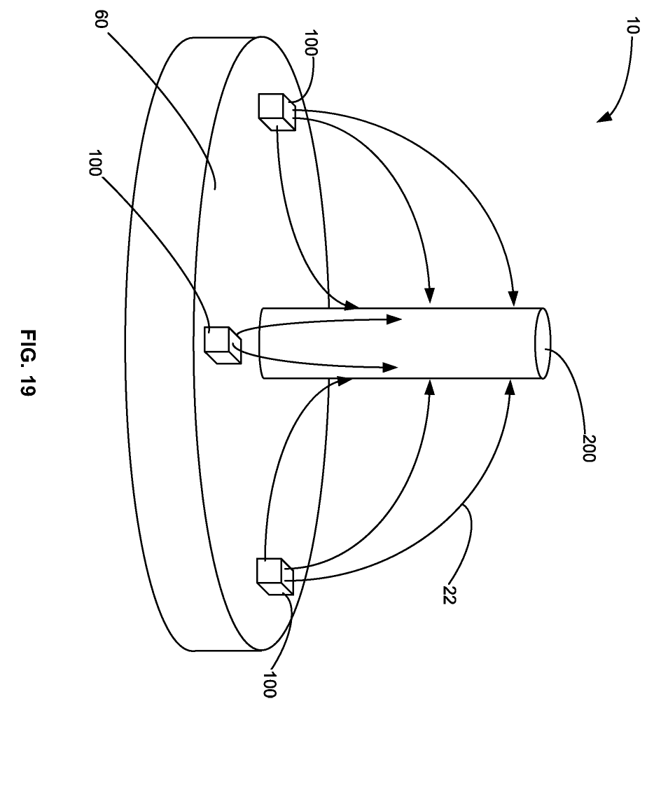

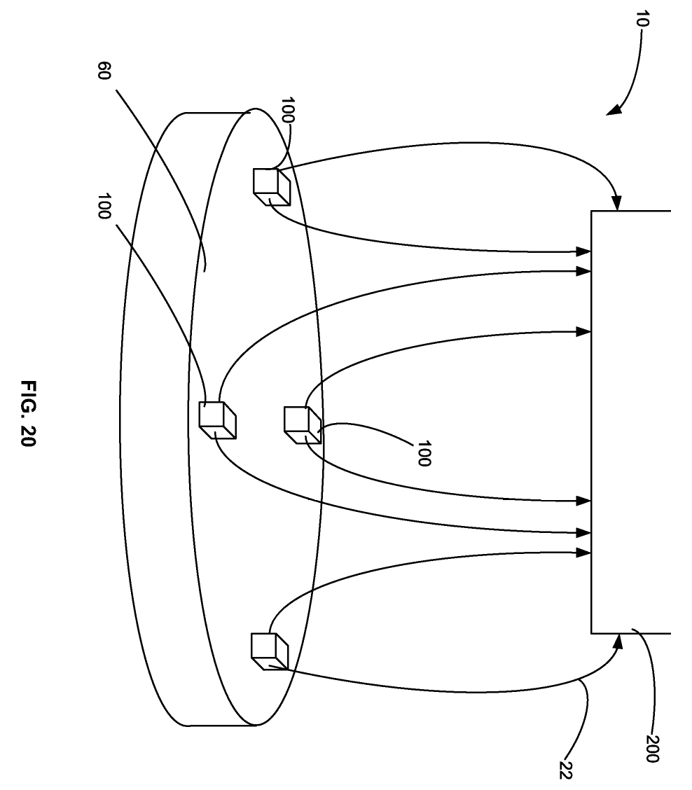

In some embodiments, the collector 200 may be placed in a central position in the enclosed space while the negative ion generators 100 are placed radially outward from the collector 200. As exemplified in FIG. 19, the collector 200 is positioned in the center the enclosed space and is shaped like a column. Having a centralized collector 200 may allow occupants in the room to be distributed along the perimeter of the enclosed space while providing a route for the negatively charged contaminants 40 to move towards the collector 200. Accordingly, the negatively charged contaminants 40 do not need to pass by an occupant of the room before being collected by the collector 200. Similarly, as exemplified in FIG. 20, the collector 200 may be positioned in a central location on the ceiling of the enclosed space. For example, in a circular classroom, students may sit along the perimeter of the enclosed space while reducing the risk of contamination to the other occupants.

It will be appreciated that the air cleaning system 10 may include a plurality of collectors 200 located in various locations in the enclosed space. For example, a first collector 200 may be positioned on the ceiling of the enclosed space while a second collector 200 may be positioned on the wall of the enclosed space. The plurality of negative ion generators 100 and the plurality of collectors 200 may be strategically positioned throughout the enclosed space for both energy and contaminate removal efficacy. For example, the plurality of negative ion generators 100 may be positioned around the perimeter of the enclosed space, as well as through the center of the enclosed space. Providing the plurality of collectors 200 on both the ceiling and/or the wall may improve the movement of the negative ions 20 to a collector 200. For example, the wall-positioned collectors 200 may collect negatively charged contaminants 40 that were charged by the negative ion generators around the perimeter of the room, while the ceiling-positioned collector 200 may collect negatively charged contaminants 40 that were charged by the negative ion generators positioned in the center of the room.

Increasing the number of collectors 200 may reduce the time between cleanings of the ceiling positioned collector 200. For example, the wall positioned collectors 200 may be easily wiped down due to their lower elevation. Similarly, as described above, the wall positioned collectors 200 may be self-cleaning, thereby requiring only intermittent cleaning of the ceiling positioned collector 200. In some embodiments, each of the plurality of collectors 200 may be self-cleaning. For example, the ceiling positioned collector 200 may use a heat source and/or UV for self-cleaning, while the wall positioned collectors 200 may include disinfecting modules 220 as described above.

### Zones

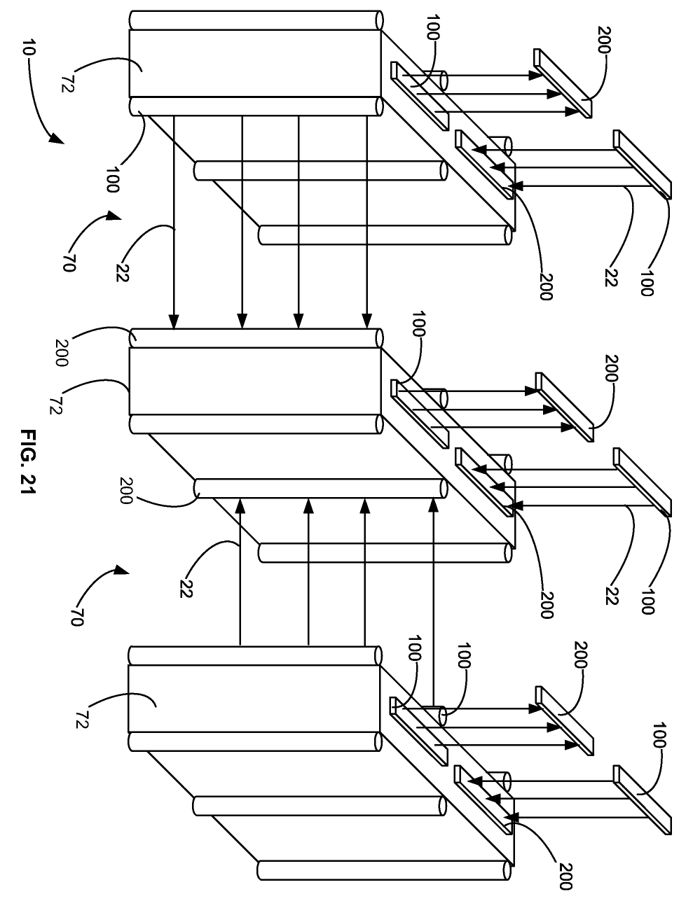

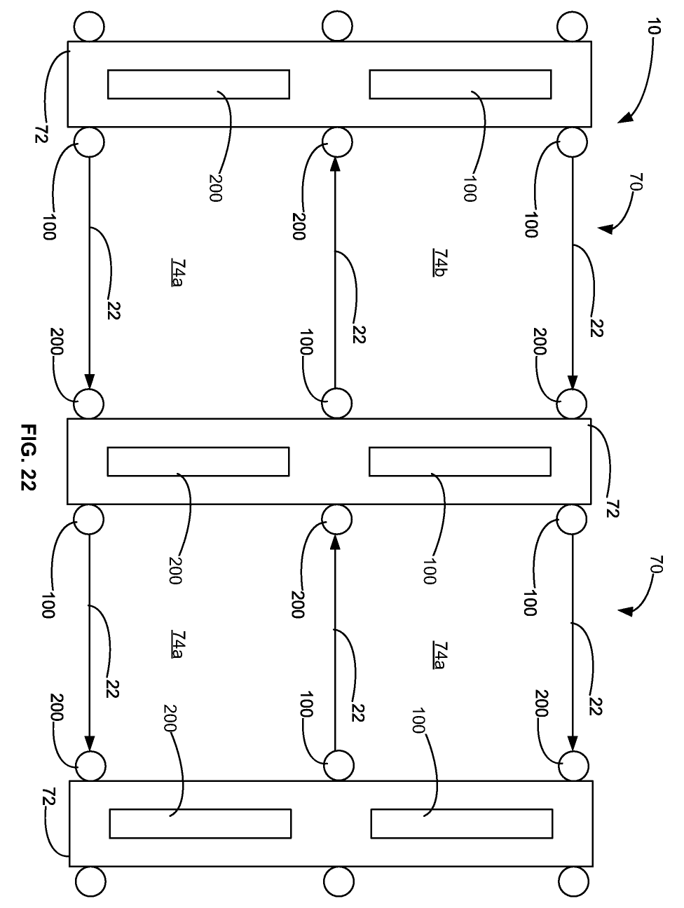

In some embodiments, the positioning of the plurality of collectors 200 and the plurality of negative ion generators 100 may be used along with social distancing principles (i.e. separating occupants by a pre-determined distance to reduce contamination). For example, as exemplified in FIGS. 21 and 22, there are a plurality of aisles 70. The aisles 70 may be in a grocery store, retail store, library, casino, office building, etc. The aisles 70 have both horizontally extending electric fields 22 and vertically extending electrical fields 22. As shown, the plurality of electric fields 22 may be used to create zones between patrons of the enclosed space. For example, the vertical electric fields 22 located on the top of shelving 72 in the aisles 70 reduces the likelihood of contaminants 30 moving from one aisle 70 to the next aisle 70. The horizontal electric fields 22 create zones 74 within the aisle 70, as is more clearly shown in FIG. 22. Accordingly, multiple occupants may be within a single aisle 70 while reducing the risk that contaminants 30 will move from one zone 74 to the next. In other words, when the first occupant leaves the first zone 74a, a second occupant may enter the first zone 74a from the second zone 74b without the risk of contaminants 30 being present in the first zone 74a.

In some embodiments, the plurality of negative ion generators 100 may each include an air moving member 110 and/or a heat source 120, as described previously. When used in the air cleaning system 10 in an enclosed space, the plurality of negative ion generators 100 may be used to generate air flow and/or convection in the enclosed space. For example, the negative ion generators 100 may be strategically positioned throughout the enclosed space to improve the efficacy of the air cleaning system 10 by coordinating the air flow and convection. In other words, the negative ion generators 100 may be used to move the negative ions 20 around the enclosed space to increase the likelihood that all contaminants 30 become negatively charged.

### Work Spaces

In some embodiments, the physical space may include a work station 62, also referred to as a work space 62, as exemplified in FIGS. 18A-18C. It will be appreciated that the work station 62 may be any working space, including, but not limited to, a work station 62 in an office, bank teller station, or a check out station in a store. As exemplified, the work station 62 includes a negative ion generator 100 and a positive collector 200. It will be appreciated that the position of the negative ion generator 100 and collector 200 may vary while still providing an electric filed between people (e.g. a customer and a clerk in a store or bank). For example, referring to FIG. 18A, the work station 62 includes a negative ion generator 100 and collector 200 at both a level of a work surface 64 and above the upper band level 11b. As exemplified in FIG. 18B, the negative ion generators 100 are at the level of the work surface 64 while the positive collectors 200 are provided above the upper band level 11b. As exemplified in FIG. 18C, the work space 62 may include one or more air moving members 110. In each embodiment, the negative ion generator 100 and collector 200 are situated to position the electric filed between people (e.g., at the front of a desk or checkout counter or the like).

In some embodiments, the negative ion generator 100 may be positioned in front of a person while working at the work station 62 and the collector 200 may be positioned overlying the person while they work at the work station 62. In some embodiments, as described previously, the positive collector 200 may be positioned below the lower band level 11a. It will be appreciated that the one or more negative ion generators 100 and one or more collectors 200 may be positioned in any location in and/or around the work station 62.

### Movable System

As described previously, the negative ion generators 100 and/or the collector 200 may be moveable, manual and/or remotely and/or automatically. For example, in some embodiments, the air cleaning system 10 may include a vacuum cleaner. The vacuum cleaner may include the negative ion generator 100 and/or the collector 200, as described previously. For example, the vacuum cleaner may automatically move around the room to distribute the negative ions 20.

In accordance with these embodiments, some or all of the collector 200 and/or the negative ion generators 100 may be located on a mobile device. As exemplified in FIG. 23, the air cleaning system 10 has a system housing 12 that includes both the negative ion generator 100 and the collector 200. As shown, the housing 12 is tubular and is located on a trolley 80 (moveable platform). The trolley 80 may be automatically movable, remotely controlled or may be manually movable. As described previously, the trolley 80 with the air cleaning system 10 may automatically move around the enclosed space using various sensors to optimize the movement pattern.

In some embodiments, the negative ion generator 100 and/or the collector 200 may include a dock to charge a moveable platform. For example, the negative ion generator 100 and collector 200 may operate as described previously, allowing, e.g., the robotic vacuum to automatically clean the floors of the enclosed space. Including the negative ion generator 100 and/or the collector 200 in the docking system for the robotic vacuum may allow for the air cleaning system 10 to be easily adapted to any enclosed space.

In some embodiments, the air cleaning system 10 may include one or more unmanned aerial vehicles (UAVs). For example, the air cleaning system 10 may include one or more drones. The drones may include the negative ion generator 100 and/or the collector 200. In some embodiments, one or more drones may include the negative ion generator 100 and one or more drones may include the collector 200. For example, a first drone may have the negative ion generator 100 and a second drone may have the collector 200. During use, the two drones may fly into the enclosed space. The first drone emits negative ions 20 that attached to contaminants 30 in the enclosed space. The second drone collects the negatively charged contaminants 40, thereby treating the air in the enclosed space. In some embodiments, the drone may include the negative ion generator 100 and the collector 200 may be positioned on the wall and/or ceiling of the enclosed space.

For example, in some embodiments, as described previously, the two drones may be used in the case of a fire. When an enclosed space is on fire, often the space fills up with smoke, making it difficult and/or dangerous for emergency responders to enter the space. The two drones may be used to fly in and clear areas of smoke from the air, thereby improving the safety of the emergency responders.

Similarly, if there has been an accident involving any manner of contaminate, such as, but not limited to, smoke, toxins, chemicals, radiation, etc., the drones and/or robotic vacuums may be used to enter the space prior to human entry and remove or reduce the level of contaminants, thereby improving the safety of responders.

In some embodiments, the robot may be used to clean the air cleaning system 10. For example, in some embodiments, the collector 200 may include a signal marker to enable the robot to clean the collector 200 based on location recognition markers.

### Systems Using a Negatively Charged Surface

In some embodiments, as described above, the negative ion generator 100 may include a surface that is negatively charged by the negative ion generator 100. For example, the surface may include, but is not limited to, a table mat, a table, a television screen, a coat, a suit, a screen, etc. Similarly, in some embodiments, the collector 200 may include a surface that is positively charged. For example, as exemplified in FIG. 9, the conductive surface 106 is negatively charged by the negative ion generator 100.

### Systems Using a wearable

In some embodiments, the negative ion generator 100 and/or the collector 200 may be wearable.

For example, the negative ion generator 100 may be a pendant that is worn around a user’s neck, as exemplified in FIG. 17. The pendent may be provided to the user as they enter, for example, a conference hall. Providing a negative ion generator 100 to each occupant in the room may reduce the likelihood of contamination by providing a local source of negative ions for the occupant. The pendent may negatively charge the occupant or the volume around the occupant. As described previously, negatively charging a surface may help repel negative ions 20, thereby improving distribution of the ions, as well as repel negatively charged contaminants 40, thereby increasing the safety of the occupant.

Alternately, or in addition, the negative ion generator 100 and/or collector 200 may be positioned on or within head apparel 90, as exemplified in FIG. 24. The negative ion generator 100 may negatively charge the head portion 92 of the head apparel 90. It will be appreciated that the negative ion generator 100 may alternately or in addition release the negative ions 20 without negatively charging the head portion 92. The collector 200 may be located, e.g., within the brim 94 of the head apparel 90. Accordingly, during use, the negative ion generator 100 releases negative ions 20 towards the front of the wearer’s face. The negative ions 20 attach to both contaminants 30 released by the wearer and by contaminants 30 that enter the space in front of the wearer’s face. The collector 200 on the brim 94 then collects the negatively charged contaminants 40.

Similarly, in some embodiments, the head apparel 90 may be a helmet or hat. The helmet 90 may include a chin strap that includes the negative ion generator 100. Accordingly, the negative ions 20 may be released below head height and may move upwardly towards the brim 94 to collect contaminants 30 in front of the wearer’s face.

Alternately or in addition, the negative ions 20 may be in a pin or lanyard and the collector 200 may be part of a hat, helmet, visor or the like, such as on the brim 94.

In some embodiments, the negative ion generator 100 and/or collector 200 may be worn as clothing. For example, an emergency responder entering the scene of an accident or fire may wear a suit that includes the negative ion generator 100.

It will be appreciated that any combination of negative ion generator 100 and/or collector 200 may be used with a wearable system and/or an automatic system. For example, a drone carrying the collector 200 may fly ahead of the occupants wearing the negative ion generator 100, thereby collecting the negatively charged contaminants in the enclosed space that have been negatively charged by the occupant’s suit. In some embodiments, the front of the suit may include the negative ion generator 100 and the back of the suit may include the collector 200. As the occupant walks into the room, the negative ion generator 100 negatively charges contaminants, which are then collected by the occupant’s back on the collector 200.

It will be appreciated that wearable air cleaning systems 10 may incorporate a heating disinfecting system, as described previously. For example, referring to the head apparel 90 in FIG. 24, the collector 200 located on the brim 94 of the head apparel 90 may include a thin outer layer and a heat source (not shown). After a period of time, the thin outer layer may rapidly heat the surface to a high temperature, killing and/or denaturing contaminants 40 collected by the collector 200. Accordingly, despite the close proximity to contaminants 40, the risk to the wearer is reduced by consistently disinfected the worn surface used as the collector 200. In some embodiments, the heating disinfecting system may be incorporated into the outer layer of clothing. During use, as the outer layer collects contaminants, the risk to the wearer is reduced by heating the outer layer of the clothing to kill and/or denature the collected contaminants 40. As described previously, the thin outer layer may be thin enough that there is no risk of burning the wearer. In some embodiments, the head apparel 90 may include a cooling system.

In some embodiments, an occupant entering the enclosed space may wear a mask to filter contaminants 30. In some embodiments, the negative ion generator 100 may be built into the mask. In some embodiments, the negative ion generator 100 may negatively charge the mask itself, thereby improving the repulsion of negatively charged contaminants 40. In some embodiments, an occupant may wear both a mask and a pendent, each having a negative ion generator 100.

### Signs

In some embodiments, the surface that is used with the collector 200 may be a sign. For example, on public transit there are often signs for advertising, information, and/or directions. The signs may be positively charged to provide a surface for use in the air cleaning system 10. Since the signs are often used in these areas, the aesthetics of the enclosed space may not be negatively affected by the use of the air cleaning system 10. Additionally, these signs may be cleaned easily to remove contaminants from the enclosed space. Such an embodiment may be used with a wearable which has a negative ion generator.

### Screens

As described previously, a screen may be used as the collection surface of the collector 200. For example, in some embodiments, a video motor and/or display may be designed to include the high voltage collector 200 in front of and/or behind the screen member. In other words, the collector 200 may be included in a screen, such as a television screen or security screen or projection screen, allowing the screen itself to act as the collecting surface of the collector 200. It will be appreciated that any aspect of the screen may be used as the collecting surface. For example, in some embodiments, the entire television may be positively charged such that the television may act as the collecting surface for the collector 200. The television may be coated with a transparent material to prevent damage from cleaning supplies and/or to improve the ease of cleaning the collecting surface. Such an embodiment may be used with a wearable which has a negative ion generator.

### Pets

In some embodiments, the air cleaning system 10 may be used in enclosed spaces with pets. For example, the collector 200 may be applied to a bed of a pet. During use, the negative ion generator 100 may negatively charge contaminants 30, such as pet dander and/or other allergens. The collector 200 may then collect the negatively charged allergens 40 to reduce the likelihood of an allergic reaction by an occupant of the enclosed space. Similarly, a litter box may be used as the collector 200 in order to reduce the disturbance of allergens.

### Lighting

In some embodiments, as described previously, the negative ion generator 100 and/or collector 200 may include, or be built into, a light source or fixture. In some embodiments, the negative ion generator 100 and/or collector 200 may include a light tube. The light tube may be used to diffuse light throughout the enclosed space, while also providing the functionality of the air cleaning system 10. For example, the collector 200 on the ceiling may include a light source, or may be positioned to accentuate a light source, while the negative ion generator 100 may be built into a lamp, or may include a light tube that can act as a lamp.

### Conference Room

As exemplified in FIG. 9, the enclosed space is a conference room with a table 60. As shown, there are three negative ion generators 100 spaced across the top of the table 60. The ceiling has a large collector 200. During use, the negative ion collectors 100 may be spaced on the table 60 to optimize the removal efficiency of biological contaminants 30 generated by the occupants of the room. In some embodiments, the collector 200 may be a part of a light fixture, as described previously.

### Restaurant

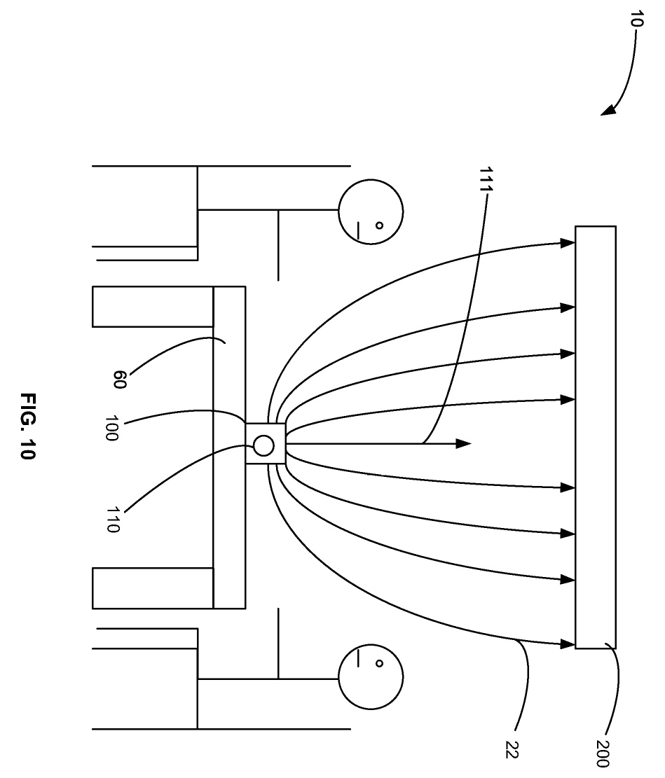

In some embodiments, as exemplified in FIG. 10, the air cleaning system 10 may be used in a restaurant. As shown, each table 60 may have a negative ion generator 100 and the ceiling has at least one collector 200. During use, occupants of the restaurant may be spaced apart a safe distance to prevent infection from one another. The negative ion generator 100 may be positioned in between the two occupants. A combination of the potential between the negatively charged contaminants 40 and the collector 200, and optionally the air moving member 110 and/or the heat source 120 may cause any exhaled, coughed, or sneezed biological contaminants 30 to be rapidly negatively charged and moved upwardly to the collector 200. Depending on the proximity between occupants, the potential may be increased or decreased to reduce the risk of infection.

In some embodiments, the air moving member 110 and/or heat source 120 may be separate from the negative ion generator 100. For example, continuing with the restaurant embodiment, the table 60 may have an air moving member 110 and/or heat source 120 built into the table. The air moving member 110 and the heat source 120 may control the air flow and convection throughout the room, such that negative ions 20 emitted from the negative ion generator 100 may be distributed effectively.

In some embodiments, such as the restaurant example, a pressure sensor may be included in the seating of the enclosed space. When an occupant sits down, the pressure sensor may indicate to the air cleaning system 10 that another occupant has entered the room and intends to stay. The air cleaning system 10 may adjust the electrical field strength accordingly and/or may initiate one or more additional negative ion generators 100 and/or collectors 200 to compensate for the increase in contaminate risk. Other sensors, such as a proximity sensor, a heat sensor or the like may be used.

### Spaces with High Ceilings

In some embodiments, as described previously, the negative ion generator 100 may be included in a negative light source and the collector 200 may be included in a positive light source. Accordingly, an array of light sources may be provided on the ceiling of the enclosed space, as exemplified in FIG. 11. The negative light sources may emit negative ions 20, which are collected by the collector 200. In some embodiments, the air moving member 110 may be used to blow the negative ions 20 down from the negative light sources, negatively charging contaminants below, before they are collected by the positive light source. In some embodiments, the ceiling may be very high, such as a high-vaulted ceiling in a church or auditorium hall. Accordingly, the air moving member 110 may blow the negative ions 20 down towards the occupants of the room (e.g. to at or near head height). The air moving member 110 may impart sufficient downward momentum such that the negative ions are directed to be within the band 11. Once the negative ions 20 negatively charge contaminants 30, the negatively charged contaminants 40 may then loop back up towards the positive light sources. Accordingly, a flow pattern may be produced of negative ions 20 being directed downwardly to a height at which people exhale, cough, or sneeze, and then the negatively charged contaminants 40 are attracted upwardly to the positive light sources.

In some embodiments, as described previously, the negative ion generator 100 and the collector 200 may be height adjustable. Accordingly, in a high-ceilinged enclosed space, the flow pattern in the space may be altered depending on the desired use of the enclosed space. For example, if a more rapid removal of contaminants is required, e.g., due to a large crowd of people, the collector 200 may be lowered closed to the occupants. In some embodiments, as described previously, the collector 200 may be built into an aesthetic aspect of the enclosed space. For example, the collector 200 may be a banner that is height adjustable. When the enclosed space becomes crowded with occupants, the banner may be lowered to improve the removal efficiency of the contaminants. It will be appreciated that the height adjustment of each of the negative ion generators 100 and/or collectors 200 may vary between each negative ion generator 100 and/or collector 200. For example, in the case of an auditorium with stadium-style seating, the negative ion generators 100 and/or collectors 200 may be lowered to match the stadium seats, such that the distance between each occupant and the negative ion generator 100 and/or collector 200 is approximately the same at each level of the stadium seating.

### Schools

In another example, the air cleaning system 10 may be used in a school. Some or all of each child’s desk may include a negative ion generator 100 and the ceiling may have one or more tiles that are collectors 200.

In some embodiments, the desk may be negatively charged by the negative ion generator 100, thereby negatively charging the child when they sit at their desk. The negatively charged surfaces may repel contaminants, as described previously.

In some embodiments, the aisle system, as exemplified in FIGS. 21 and 22 may be used in a school. For example, each child’s desk may have a zone 74 wherein the child is the only occupant within the zone 74. Accordingly, the risk of cross-contamination between zones 74 is reduced. Similarly, air moving members 110 may be used to form air barriers, as described previously, to further reduce the risk of cross contamination.

### Casinos

In another example, casinos may use the air cleaning system 10. For example, the aisle system as illustrated in FIGS. 21 and 22 may be used to set broad zones 74 within the casino. Individual tables, such as for poker or blackjack, may have their own air cleaning system 10, optionally with a negatively charged table top to repel negative ions 20 up towards a collector 200 located above the table. Similarly, slot machines may each have a collector located on or above the screen, with one or more negative ion generators 100 located around the seating area.

### Transportation Vehicles

In another example, airplanes, cars, and trains may use the air cleaning system 10. Each seat of the vehicle may include a negative ion generator 100, and the roof of the vehicle may have one or more collectors 200.

### Residential Homes

In another example, the air cleaning system 10 may be used in a residential home. Negative ion generators 100 may be positioned around the home, with the collector 200 located on the ceiling, behind the television, on the television, on the wall, etc.

### Radiation

In another example, the air cleaning system 10 may be used in enclosed spaces that are exposed to radiation. Particles in the enclosed space may become radioactive with exposure to radiation. Negatively charging the radioactive particles in the room may reduce the levels of radiation exposure to occupants.

While the above description describes features of example embodiments, it will be appreciated that some features and/or functions of the described embodiments are susceptible to modification without departing from the spirit and principles of operation of the described embodiments. For example, the various characteristics which are described by means of the represented embodiments or examples may be selectively combined with each other. Accordingly, what has been described above is intended to be illustrative of the claimed concept and non-limiting. It will be understood by persons skilled in the art that other variants and modifications may be made without departing from the scope of the invention as defined in the claims appended hereto. The scope of the claims should not be limited by the preferred embodiments and examples, but should be given the broadest interpretation consistent with the description as a whole.

What is claimed is:

1. A method for removing contaminants from air in a physical space wherein, when a person is in the physical space, airflow is provided in the physical space and a head of the person is positioned in a band extending from a lower band level, wherein the head of the person is positioned above the lower band level during normal use of the physical space, and an upper band level, wherein the head of the person is positioned at least partially below the upper band level during normal use of the physical space, the method comprising:
providing negatively charged ions in the physical space;
operating a positively charged ion collector to produce an electrical field, the electrical field having an electrical field strength, wherein the negatively charged ions in the band are drawn towards the positively charged ion collector due to a potential difference between the negatively charged ions and the positively charged ion collector; and
adjusting the electrical field strength whereby negatively charged ions travel to the positively charged ion collector regardless of the airflow in the physical space.

2. The method of claim 1 wherein the airflow is resolvable into a first flow vector directed at the positively charged ion collector and a second flow vector directed away from the positively charged ion collector, wherein the electrical field strength is adjusted such that a force imparted to the air by the second flow vector is less than the field strength of the attractive force.

3. The method of claim 2 wherein the electrical field strength is adjusted by at least one of changing a voltage of a negative ion generator, changing a voltage of the positively charged ion collector, changing a position of the negative ion generator and changing a position of the positively charged ion collector.

4. The method of claim 1 further comprising adjusting the field strength based on a signal provided by one or more sensors.

5. The method of claim 1 further comprising providing a sensor that issues a signal wherein the signal is indicative of a value of at least one of an electric field strength, a concentration level of airborne contaminants within the physical space, a negative ion concentration level within the physical space, a negative ion generator voltage, and a positively charged ion collector voltage falling outside of a threshold level and adjusting the field strength based on the signal provided by the sensor.

6. The method of claim 2 adjusting the airflow whereby the first flow vector comprises at least 70% of the force imparted to the air.

7. The method of claim 1 further comprising adjusting at least one of a negative ion generator and the positively charged ion collector to maintain a generally constant field strength as a location of the person in the physical space varies.

8. The method of claim 1 further comprising disinfecting an outer surface of the positively charged ion collector such that viruses on the outer surface of the positively charged ion collector are denatured.

9. The method of claim 1 further comprising adjusting at least one of a position of the positively charged ion collector and a position of a negative ion generator during operation of the positively charged ion collector and the negative ion generator.

10. The method of claim 1 further comprising, prior to providing the negatively charged ions in the physical space, heating the negatively charged ions.

11. The method of claim 1 further comprising adjusting the electrical field strength such that the negatively charged ions in the physical space are drawn into the band.

12. A method for removing contaminants from air in a physical space wherein, when a person is in the physical space, airflow is provided in the physical space and a head of the person is positioned in a band extending from a lower band level, wherein the head of the person is positioned above the lower band level during normal use of the physical space, and an upper band level, wherein the head of the person is positioned at least partially below the upper band level during normal use of the physical space, the method comprising:
providing negatively charged ions in the physical space;
operating a positively charged ion collector to produce an electrical field, the electrical field having an electrical field strength, wherein the negatively charged ions in the band are drawn towards the positively charged ion collector due to a potential difference between the negatively charged ions and the positively charged ion collector; and
adjusting the airflow such that at all times the air flows towards the positively charged ion collector.

13. The method of claim 12 further comprising adjusting the airflow by at least one of changing a direction of the airflow and changing a velocity of the airflow whereby at all times the air flows towards the positively charged ion collector.

14. The method of claim 12 further comprising increasing the electric field strength by at least one of changing a voltage of a negative ion generator, changing a voltage of the positively charged ion collector, changing a position of the negative ion generator, and changing a position of the positively charged ion collector.

15. The method of claim 12, further comprising adjusting the airflow such that the negatively charged ions in the physical space flow into the band.

16. A method for removing contaminants from air in a work space, wherein when a person is in the work space, airflow is provided in the work space and a head of the person is positioned in a band extending from a lower band level, wherein the head of the person is positioned above the lower band level during normal use of the work space, and an upper band level, wherein the head of the person is positioned at least partially below the upper band level during normal use of the work space, the method comprising:
providing negatively charged ions in the workspace;
operating a positively charged ion collector positioned above the upper band level to produce an electrical field, the electrical field having an electrical field strength, wherein the negatively charged ions in the band are drawn towards the positively charged ion collector due to a potential difference between the negatively charged ions and the positively charged ion collector; and
adjusting the airflow such that the airflow does not prevent the negatively charged ions from travelling to the positively charged ion collector.

17. The method of claim 16 further comprising adjusting the airflow by at least one of changing a direction of the airflow and changing a velocity of the airflow.

18. The method of claim 16 wherein the first zone is at a location in front of a negative ion generator, and the second zone is at a location of the person which is rearward of the negative ion generator and the method further comprises adjusting the airflow such that the airflow inhibits contaminants from passing tangentially through the airflow from a first zone of air into a second zone of air.

19. The method of claim 16 further comprising adjusting the electric field strength such that the flow of negatively charged ions towards the positively charged ion collector inhibits contaminants from passing tangentially through the flow of negatively charged ions from the first zone of air into the second zone of air.
